(19) United States
(12) Patent Application Publication
(10) Pub. No.: US 2005/0271616 A1
(43) Pub. Date: Dec. 8, 2005

(54) Novel block polymers, compositions comprising them, and processes for treating kerating materials therewith

(76) Inventors: Timo Luukas, Massy (FR); Celine Farcet, Paris (FR)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/087,990

(22) Filed: Mar. 24, 2005

Related U.S. Application Data
(60) Provisional application No. 60/559,968, filed on Apr. 7, 2004.

(30) Foreign Application Priority Data
Mar. 26, 2004 (FR) 04 03185

(51) Int. Cl. A61K 31/785

(52) U.S. Cl. 424/78.3; 525/205

(57) ABSTRACT

The present disclosure relates to novel block polymers comprising at least one monomer with an optical effect of formula (I), and to cosmetic compositions comprising them, such as nail varnish compositions, foundation compositions and anti-ageing compositions. The present disclosure also relates to a cosmetic treatment process comprising the application of the composition to keratin materials.

This application claims benefit of U.S. Provisional Application No. 60/559,968, filed Apr. 7, 2004, and French Application No. 04/03185 filed Mar. 28, 2005, the contents of both of which are incorporated herein by reference.

The present disclosure relates to novel polymers with a specific structure, and to cosmetic compositions comprising the polymers. The present disclosure also relates to a cosmetic treatment process using the polymers as described herein.

Various types of polymers have conventionally been used in cosmetic compositions on account of the various properties that they may give to these compositions. They may be used, for example, in makeup or care compositions for the skin, the lips or the integuments, such as nail varnishes or hair care compositions. However, when using two polymers that are incompatible, i.e. immiscible in the same solvent, within the same composition, the formulator may be confronted, as a result of the incompatibility of the polymers, with problems of phase separation or even of decantation, and in general with the production of a non-uniform composition. Previously, these problems could usually only be solved by the presence in the composition of a compound for rendering the polymers mutually compatible.

Thus, in one aspect, the present disclosure relates to a polymer which, when included in a composition, for example, a cosmetic composition, can enable the composition to avoid the drawbacks, limitations, defects and disadvantages of the compositions of the prior art. In particular, disclosed herein is a polymer, referred to as a block polymer, comprising at least one first block and at least one second block that are incompatible with each other and that can have, for instance, different glass transition temperatures (Tg), wherein the first and second blocks are linked together via an intermediate segment comprising at least one constituent monomer of the first block and at least one constituent monomer of the second block.

Thus, the present disclosure relates to a polymer as defined below, and also to a composition, for instance a cosmetic composition, comprising the polymer.

It has been found, surprisingly, that the polymers according to the present disclosure can have good optical properties that make it possible to use them in cosmetics, for obtaining adequate optical effects for the compositions comprising them and/or for the makeup obtained using these compositions.

Depending on the nature of the substituents, they may show large variability in color, which may range from yellow to red/violet. This can make it possible to have a range of compounds, belonging to the same chemical family and thus being formulated in a similar manner, which can provide a diversity of optical properties; this can, for example, facilitate the work of formulators by allowing them to keep a common architecture for all of their compositions, irrespective of the polymers with an optical property that are used.

Moreover, the polymers according to the present disclosure can have good fluorescence properties. It is recalled that fluorescent compounds absorb in the ultraviolet and visible range, and re-emit energy by fluorescence with a wavelength ranging from 380 nm to 830 nm.

The polymers according to the present disclosure may be in solid or liquid form, and can give noteworthy-optical effects to the compositions comprising them and also to the makeup applied;for instance, they can provide lightening effects, illuminating effects and/or color effects. Moreover, these polymers may show good temperature, pH and/or light stability.

It has also been found that the polymers according to the present disclosure show good solubility in fatty substances, it being possible for this solubility to vary and to be adjusted, according to the nature of the monomers. This good liposolubility may also facilitate their subsequent use, for example in cosmetic compositions generally comprising a fatty phase.

As used herein, the term “at least one block” is understood to mean one or more blocks.

As used herein, the term “mutually incompatible blocks” is understood to mean that the mixture formed from the polymer corresponding to the at least one first block and from the polymer corresponding to the at least one second block is immiscible in the polymerization solvent that is in weight majority for the block polymer, at room temperature (25° C.) and atmospheric pressure (10⁵ Pa), for a polymer mixture amount of greater than or equal to 5% by weight, relative to the total weight of the mixture (polymers and solvent), it being understood that:

i) the polymers are present in the mixture in an amount such that the respective weight ratio ranges from 10/90 to 90/10, and that

ii) each of the polymers corresponding to the at least one first and second blocks has an average (weight-average or number-average) molecular mass equal to that of the block polymer ±15%.

When there is a mixture of polymerization solvents, that is, when two or more solvents are present, the polymer mixture is immiscible in at least one of them.

Needless to say, in the case of a polymerization performed in a single solvent, this solvent is the solvent that is in majority.

The intermediate segment is a block comprising at least one constituent monomer ml of the first block and at least one constituent monomer m2 of the second block of the polymer; for instance, in one embodiment, m2 is different from m1. The intermediate segment or block can allow these first and second blocks to be “compatibilized”.

By incorporating these novel polymers into cosmetic compositions, it has been discovered that some of these polymers described in greater detail hereinbelow, surprisingly, can have beneficial cosmetic properties.

In general, these polymers may increase the impact strength of nail varnishes and improve the staying power of a wide variety of makeup compositions, for example, foundations or lipsticks, without causing the user any sensation of discomfort. They may moreover have tensioning properties.

The block polymer of the composition according to the present disclosure can be, for instance, a linear block ethylenic polymer, for example, a polymer capable of forming a deposit, such as a film-forming polymer.

As used herein, the term “ethylenic polymer” is understood to mean a polymer obtained by polymerization of monomers comprising an ethylenic unsaturation.

As used herein, the term “block polymer” is understood to mean a polymer comprising at least two different blocks, for example, at least 3 different blocks.

The polymer is a polymer of linear structure, as opposed to a polymer of non-linear structure, which is, for example, a polymer of branched, starburst or grafted structure, or the like.

As used herein, the term “polymer forming a deposit” is understood to mean a polymer capable, by itself or in the presence of an auxiliary agent, of forming a deposit that adheres to a support, such as to keratin materials.

As used herein, the term “film-forming polymer” is understood to mean a polymer that is capable, by itself or in the presence of an auxiliary film-forming agent, of forming a continuous film that adheres to a support, such as to keratin materials.

The polymer according to the present disclosure comprises at least one first block and at least one second block that are incompatible with each other and that can have, for instance, different glass transition temperatures (Tg), wherein the first and second blocks are linked together via an intermediate segment comprising at least one constituent monomer of the first block and at least one constituent monomer of the second block.

It must be noted that, in the text hereinabove and hereinbelow, the terms “first” and “second” blocks do not in any way condition the order of the blocks in the structure of the polymer.

Each block of the polymer according to the present disclosure can be derived from one type of monomer or from several different types of monomer. This means that each block may comprise a homopolymer or a copolymer; this copolymer constituting the block may in turn be random or alternating.

For example, the intermediate segment comprising at least one constituent monomer of the first block and at least one constituent monomer of the second block of the polymer can be a random polymer. For instance, the intermediate block can essentially be derived from constituent monomers of the first block and of the second block. As used herein, the term “essentially” is understood to mean at least 85%, such as at least 90%, for example, 95% and 100%.

The intermediate block can have, for example, a glass transition temperature Tg that is.between the glass transition temperatures of the first and second blocks.

According to one aspect of the present disclosure, the first and second blocks can have different glass transition temperatures, for instance, with a difference between the glass transition temperatures of the first and second blocks of greater than 5° C., such as greater than 10° C., and for example, greater than 20° C.

The glass transition temperatures indicated for the at least one first and second blocks may be theoretical Tg values determined from the theoretical Tg values of the constituent monomers of each of the blocks, which may be found in a reference manual such as the Polymer Handbook, 3rd Edition, 1989, John Wiley, according to the following relationship, known as Fox's law:

$$\frac{1}{Tg} = {\sum }_{i} ( \frac{\overline{ω} ⅈ}{Tgi} )$$

wherein \overline{ω}i is the mass fraction of the monomer i in the block under consideration and Tgi is the glass transition temperature of the homopolymer of the monomer i. Unless otherwise indicated, the Tg values indicated for the at least one first and second blocks in the present disclosure are theoretical Tg values.

In the compound according to the present disclosure, at least one of the blocks comprises at least one monomer hereinbelow referred to as a monomer “with an optical effect,” which is chosen from those of formula (I): 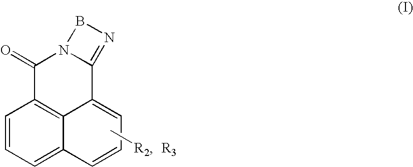
wherein:

R₂ and R₃, which may be present on the same ring or each on a different ring, are independently chosen from hydrogen atoms, halogen atoms, and groups of formula (II): —X-G-P, with the proviso that at least one of the radicals R₂ and/or R₃ is a group of formula (II), wherein:
X is chosen from oxygen and sulfur atoms, and —SO—, —SO₂—, —NH— and —NR— groups, wherein R is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms; and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms, optionally substituted with at least one entity chosen from ═O, OH, and NH₂ groups and halogen atoms; and/or optionally interrupted with at least one heteroatom chosen from O, N, P, Si and S atoms;
P is a polymerizable group chosen from those of formulae (IIIa) to (IIIc): 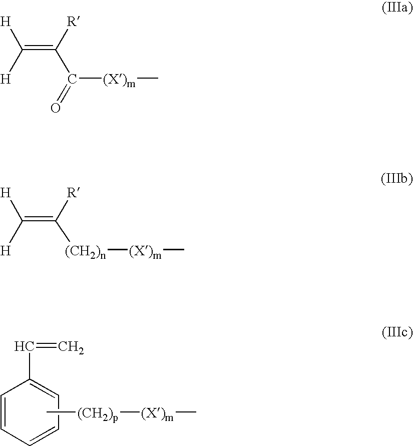
   wherein:
R¹ is chosen from a hydrogen atom and linear and branched, saturated C₁₋₆ hydrocarbon-based radicals,
X′ is chosen from an oxygen atom, and NH and NR″ groups, wherein R″ is chosen from C1-6 alkyl, C6-10 aryl, (C6-10)aryl(C1-6)alkyl and (C1-6)alkyl(C6-10)aryl radicals, the alkyl and/or aryl groups also possibly being substituted with at least one entity chosen from halogen atoms and OH, C1-6 alkoxy, and C6-10 aryloxy groups; in one embodiment of the present disclosure, X′ is oxygen;
m is equal to 0 or 1; n is equal to 0 or 1; p is equal to 0, 1 or 2;

B is a divalent aromatic group chosen from those of formulae (IVa) to (IVd): 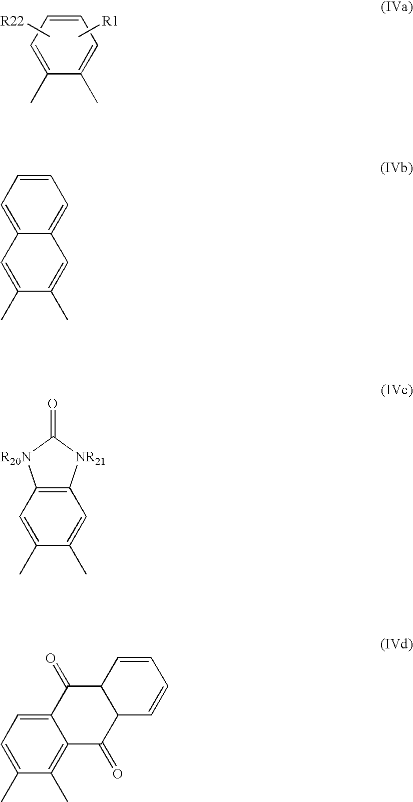
wherein:

R1 is chosen from linear, branched and/or cyclic, saturated and/or unsaturated carbon-based radicals comprising from 1 to 32 carbon atoms, optionally substituted with at least one entity chosen from ═O, OH, and NH₂ groups, and halogen atoms; and/or optionally interrupted with at least one heteroatom chosen from O, N, P, Si and S atoms;

R22 is chosen from a hydrogen atom and linear, branched and/or cyclic, saturated and/or unsaturated carbon-based radicals comprising from 1 to 32 carbon atoms, optionally substituted with at least one entity chosen from ═O, OH, and NH₂ groups and halogen atoms; and/or optionally interrupted with at least one heteroatom chosen from O, N, P, Si and S atoms;

R20 and R21, which may be identical or different, are chosen from hydrogen atoms, and linear and branched C1-8 alkyl radicals and cyclopentyl, cyclohexyl, cyclooctyl, cyclodecyl, cyclododecyl, benzyl, naphthyl and phenyl radicals.

As used herein, the term “cyclic radical” is understood to mean a monocyclic or polycyclic radical, which is itself thus in the form of at least one saturated and/or unsaturated, optionally substituted ring (for example cyclohexyl, cyclodecyl, benzyl or fluorenyl), but also a radical comprising at least one of the rings (for example p-tert-butylcyclohexyl or 4-hydroxybenzyl).

As used herein, the term “saturated and/or unsaturated radical” is understood to mean totally saturated radicals, totally unsaturated radicals, including aromatic radicals, and also radicals comprising at least one double and/or triple bond, the rest of the bonds being single bonds.

In one embodiment of the present disclosure, R2 is a hydrogen atom and R3 is thus a group of formula (II).

In the group of formula (II), X may be chosen from oxygen atoms, and —NH— and —NR— radicals, wherein R may, for instance, be chosen from linear, branched and/or cyclic, saturated and unsaturated hydrocarbon-based radicals, optionally comprising a hydrocarbon-based ring that is itself saturated or unsaturated, comprising from 2 to 18, such as from 3 to 12 carbon atoms, optionally substituted with at least one group chosen from ═O, OH, and NH₂ groups, and halogen atoms; and/or optionally interrupted with at least one heteroatom chosen from O, N, P, Si and S atoms.

For example, in one embodiment, R may be chosen from ethyl, n-propyl, isopropyl, n-butyl, isobutyl, tert-butyl, pentyl, hexyl, cyclohexyl, octyl, cyclooctyl, decyl, cyclodecyl, dodecyl, cyclodecyl, phenyl and benzyl radicals.

In another embodiment, X may be chosen, for example, from —NH— and —NR— radicals, wherein R is cyclohexyl. In still another embodiment, when X is equal to NR, then, for example, B may be different from formula (IVa).

The divalent radical G may be chosen from, for instance, linear, branched and/or cyclic, saturated and unsaturated divalent hydrocarbon-based radicals, optionally comprising a hydrocarbon-based ring that is itself saturated or unsaturated, comprising, in total, from 2 to 18, such as from 3 to 10 carbon atoms, optionally substituted with at least one group chosen from ═O, OH, and NH₂ groups, and halogen atoms; and/or optionally interrupted with at least one heteroatom chosen from O, N, P and Si atoms. For example, G may be chosen from linear and branched, saturated divalent hydrocarbon-based radicals optionally comprising a saturated hydrocarbon-based ring, comprising in total from 2 to 18, such as from 3 to 10 carbon atoms. As a further example, in one embodiment of the present disclosure, G may be chosen from ethylene, n-propylene, isopropylene, 1-methylethylene, 2-methylethylene, n-butylene, isobutylene, pentylene, such as n-pentylene, hexylene, for instance n-hexylene, cyclohexylene, heptylene, octylene, cyclooctylene, decylene, cyclodecylene, cyclohexyldimethylene, such as those of formula —CH₂—C₆H₁₀—CH₂—, dodecylene and cyclododecylene radicals.

For instance, in another embodiment of the present disclosure, in the formula (IIIb), if n=0 then m=0.

The polymerizable group P, for example, may be chosen from at least one of the formulae: 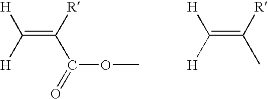
wherein R′ is chosen from a hydrogen atom and a methyl group.

The group B, for instance, may be chosen from those of formula (IVa) wherein R1 may be chosen from linear, branched and/or cyclic, saturated carbon-based radicals comprising from 1 to 32 carbon atoms, for example from 2 to 12 and from 3 to 6 carbon atoms; for further instance, R1 may be chosen from methyl, ethyl and propyl radicals.

Among the monomer compounds that may be used according to the present disclosure, non-limiting mention may be made of the compounds of the following formulae, wherein R is a hydrogen atom or a methyl group:

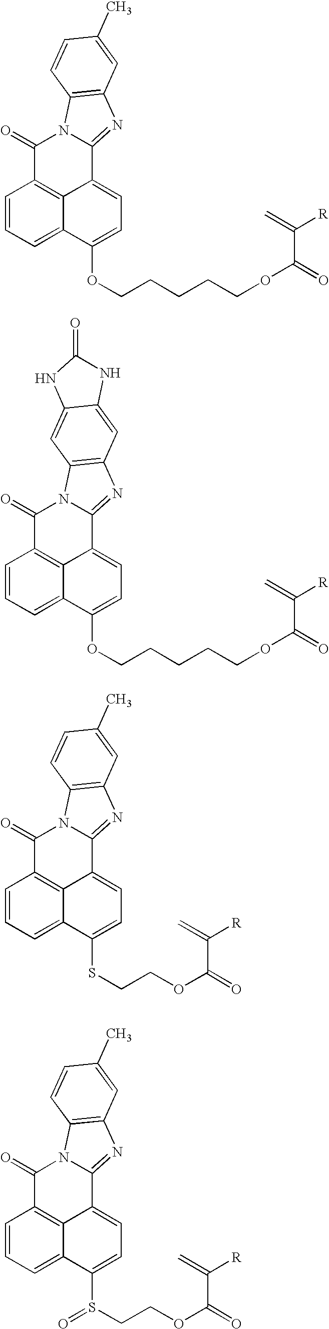

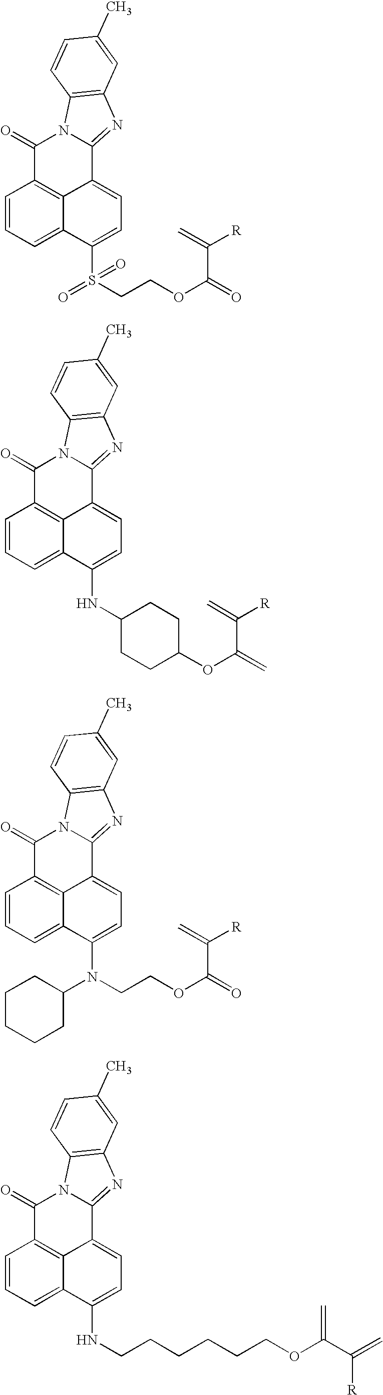

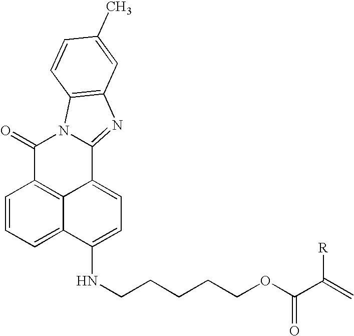

This monomer can be present, alone or as a mixture, in the at least one first and/or at least one second block.

The block comprising the monomer with an optical effect of formula (I) may thus be:

(i) a homopolymer comprising only one monomer with an optical effect of formula (I),

(ii) a copolymer comprising several monomers with an optical effect of formula (I),

(iii) a copolymer comprising at least one monomer with an optical effect of formula (I), and at least one additional monomer, which may be chosen, for example, from the monomers with an optical effect of formulae (A), (B) and/or (C) below and the “usual” additional monomers known in the art.

Among the additional monomers that may be present in the block comprising the at least one monomer with an optical effect of formula (I), and/or which may be present in the at least one other block not comprising a monomer with an optical effect of formula (I), non-limiting mention may be made, alone or as a mixture, of the following monomers:

(i) ethylenic hydrocarbons comprising from 2 to 10 carbons, such as ethylene, isoprene or butadiene;

(ii) the (meth)acrylates of formulae: 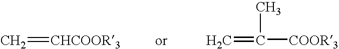
wherein R′₃ is chosen from:

linear and branched alkyl groups of 1 to 18 carbon atoms, optionally intercalated with at least one heteroatom chosen from O, N, S and P atoms; the alkyl group also possibly being optionally substituted with at least one substituent chosen from hydroxyl groups, halogen atoms (such as Cl, Br, I and F), and groups Si(R₄R₅), wherein R₄ and R₅, which may be identical or different, are chosen from C₁ to C₆ alkyl groups and phenyl groups; for example, R′₃ may be chosen from methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl or stearyl group; 2-ethylperfluorohexyl; or a C₁₋₄ hydroxyalkyl group such as 2-hydroxyethyl, 2-hydroxybutyl or 2-hydroxypropyl; or a (C₁₋₄)alkoxy(C₁₋₄)alkyl group such as methoxyethyl, ethoxyethyl or methoxypropyl,

C₃ to C₁₂ cycloalkyl groups such as an isobornyl group,

C₃ to C₂₀ aryl groups such as a phenyl group,

C₄ to C₃₀ aralkyl groups (with C₁ to C₈ alkyl groups) such as 2-phenylethyl, t-butylbenzyl or benzyl,

4- to 12-membered heterocyclic groups comprising at least one heteroatom chosen from O, N and S atoms, the ring being aromatic or non-aromatic,

heterocycloalkyl groups (with C1 to C4 alkyl), such as furfurylmethyl or tetrahydrofurfurylmethyl,

wherein the cycloalkyl, aryl, aralkyl, heterocyclic or heterocycloalkyl groups may be optionally substituted with at least one substituent chosen from hydroxyl groups, halogen atoms, and linear and branched C₁₋₄ alkyl groups which can be optionally intercalated with at least one heteroatom chosen from O, N, S and P atoms, the alkyl groups also possibly being optionally substituted with at least one substituent chosen from hydroxyl groups, halogen atoms (such as Cl, Br, I and F), and groups Si(R₄R₅), wherein R₄ and R₅, which may be identical or different, are chosen from C₁ to C₆ alkyl groups and phenyl groups,

R′₃ may also be a group —(C₂H₄O)ₘ—R″, wherein m ranges from 5 to 150 and R″ is chosen from a hydrogen atom and C₁ to C₃₀ alkyl groups, for example —POE-methyl or —POE-behenyl;

(iii) the (meth)acrylamides of formula: 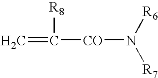
wherein R₈ is chosen from a hydrogen atom and a methyl group; and R₇ and R₆, which may be identical or different, are chosen from hydrogen atoms, and linear and branched alkyl groups of 1 to 18 carbon atoms, optionally intercalated with at least one heteroatom chosen from O, N, S and P; the alkyl groups also possibly being optionally substituted with at least one-substituent chosen from hydroxyl groups, halogen atoms (such as Cl, Br, I and F), and groups. Si(R₄R₅), wherein R₄ and R₅, which may be identical or different, are chosen from C₁ to C₆ alkyl groups and phenyl groups; for example, R₆ and/or R₇ may be chosen from methyl, ethyl, propyl, n-butyl, isobutyl, tert-butyl, hexyl, ethylhexyl, octyl, lauryl, isooctyl, isodecyl, dodecyl, cyclohexyl, t-butylcyclohexyl and stearyl groups; 2-ethylperfluorohexyl; and C₁₋₄ hydroxyalkyl groups such as 2-hydroxyethyl, 2-hydroxybutyl and 2-hydroxypropyl; and (C₁₋₄)alkoxy(C₁₋₄)alkyl-groups such as methoxyethyl, ethoxyethyl and methoxypropyl;

C₃ to C₁₂ cycloalkyl groups, such as an isobornyl group,

C₃ to C₂₀ aryl groups such as a phenyl group,

C₄ to C₃₀ aralkyl groups (C₁ to C₈ alkyl groups) such as 2-phenylethyl, t-butylbenzyl and benzyl,

4- to 1 2-membered heterocyclic groups comprising at least one heteroatom chosen from O, N and S atoms, the ring being aromatic or non-aromatic,

heterocycloalkyl groups (C1 to C4 alkyl groups), such as furfurylmethyl or tetrahydrofurfurylmethyl,

wherein the cycloalkyl, aryl, aralkyl, heterocyclic and/or heterocycloalkyl groups are optionally substituted with at least one substituent chosen from hydroxyl groups, halogen atoms, and linear and branched C₁-C₄ alkyl groups which can be optionally intercalated with at least one heteroatom chosen from O, N, S and P atoms, the alkyl groups also being optionally substituted with at least one substituent chosen from hydroxyl groups, halogen atoms (such as Cl, Br, I and F) and groups Si(R₄R₅), wherein R₄ and R₅, which may be identical or different, are chosen from C₁ to C₆ alkyl groups and phenyl groups.

Non-limiting examples of (meth)acrylamide monomers that may be used are (meth)acrylamide, N-ethyl(meth)acrylamide, N-butylacrylamide, N-t-butylacrylamide, N-isopropylacrylamide, N,N-dimethyl(meth)acrylamide, N,N-dibutylacrylamide, N-octylacrylamide, N-dodecylacrylamide, undecylacrylamide and N(2-hydroxypropylmethacrylamide).

(iv) the vinyl compounds of formula:
CH₂═CH—R₉, CH₂═CH—CH₂—R₉ or CH₂═C(CH₃)—CH₂—R₉
wherein R₉ is chosen from hydroxyl groups, halogen atoms (Cl or F), NH₂ groups, and OR₁₄ groups, in which R₁₄ is chosen from phenyl groups and C₁ to C₁₂ alkyl groups (the monomer is a vinyl or allylic ether); acetamide (NHCOCH₃) groups; OCOR₁₅ groups in which R₁₅ is chosen from linear and branched alkyl groups of 2 to 12 carbons (the monomer is a vinyl or allylic ester); and groups chosen from:

linear and- branched alkyl groups of 1 to 18 carbon atoms optionally intercalated with at least one heteroatom chosen from O, N, S and P atoms; the alkyl group also being optionally substituted with at least one substituent chosen from hydroxyl groups, halogen atoms (such as Cl, Br, I and F) and groups Si(R₄R₅), in which R₄ and R₅, which may be identical or different, are chosen from C₁ to C₆ alkyl groups and phenyl groups;

C₃ to C₁₂ cycloalkyl groups, such as isobornyl or cyclohexane,

C₃ to C₂₀ aryl groups, such as phenyl,

C₄ to C₃₀ aralkyl groups (C₁ to C₈ alkyl group) such as 2-phenylethyl; benzyl,

4- to 12-membered heterocyclic groups comprising at least one heteroatom chosen from O, N and S atoms, the ring being aromatic or non-aromatic,

heterocycloalkyl groups (C1 to C4 alkyl), such as furfurylmethyl or tetrahydrofurfurylmethyl,

wherein the cycloalkyl, aryl, aralkyl, heterocyclic and heterocycloalkyl groups are optionally substituted with at least one substituent chosen from hydroxyl groups, halogen atoms, and linear and branched C1 to C4 alkyl groups optionally intercalated with at least one heteroatom chosen from O, N, S and P atoms, the alkyl groups also being optionally substituted with at least one substituent chosen from hydroxyl groups, halogen atoms (Cl, Br, I and F) and groups Si(R₄R₅) in which R₄ and R₅, which may be identical or different, are chosen from C₁ to C₆ alkyl groups, and phenyl groups.

Non-limiting examples of vinyl monomers are vinyl cyclohexane and styrene. Non-limiting examples of vinyl esters are vinyl acetate, vinyl propionate, vinyl butyrate, vinyl ethylhexanoate, vinyl neononanoate and vinyl neododecanoate.

Among the vinyl ethers that may be used as disclosed herein, non-limiting mention may be made of methyl vinyl ether, ethyl vinyl ether and isobutyl vinyl ether.

(v) (meth)acrylic, (meth)acrylamide or vinyl monomers comprising a fluoro or perfluoro group, such as ethylperfluorooctyl or 2-ethylperfluorohexyl (meth)acrylate;

(vi) silicone-based (meth)acrylic, (meth)acrylamide or vinyl monomers, such as methacryloxypropyltris(trimethylsiloxy)silane or acryloxypropylpolydimethylsiloxane;

(vii) ethylenically unsaturated monomers comprising at least one carboxylic, phosphoric or sulfonic acid, or anhydride functional group, for instance acrylic acid, methacrylic acid, crotonic acid, maleic anhydride, itaconic acid, fumaric acid, maleic acid, acrylamidopropanesulfonic acid, vinylbenzoic acid and vinylphosphoric acid, and the salts thereof;

(viii) ethylenically unsaturated monomers comprising at least one tertiary amine functional group, for instance 2-vinylpyridine, 4-vinylpyridine, dimethylaminoethyl methacrylate, diethylaminoethyl methacrylate and dimethylaminopropylmethacrylamide, and the salts thereof.

The salts may be formed by neutralization of the anionic groups with a mineral base, such as LiOH, NaOH, KOH, Ca(OH)₂, NH₄OH or Zn(OH)₂; or with an organic base such as a primary, secondary or tertiary alkylamine, such as triethylamine or butylamine. This primary, secondary or tertiary alkylamine may comprise at least one nitrogen and/or oxygen atoms and may thus comprise, for example, at least one or more alcohol functional groups; non-limiting mention may be made for example, of amino-2-methyl-2-propanol, triethanolamine and dimethylamino-2-propanol. Non-limiting mention may also be made of lysine or 3-(dimethylamino)propylamine.

Further non-limiting mention may also be made of the salts of mineral acids, such as sulfuric acid, hydrochloric acid, hydrobromic acid, hydriodic acid, phosphoric acid or boric acid. Non-limiting mention may also be made of the salts of organic acids, which may comprise at least-one carboxylic, sulfonic or phosphonic acid group. They may be linear, branched, or cyclic aliphatic acids, or alternatively aromatic acids. These acids may also comprise at least one heteroatom chosen from O and N atoms, for example in the form of hydroxyl groups. Non-limiting mention may be made for instance of propionic acid, acetic acid, terephthalic acid, citric acid and tartaric acid.

The additional comonomers can be, for example, chosen, alone or as a mixture, from C₁-C₁₈ alkyl or C₃-C₁₂ cycloalkyl (meth)acrylates, such as from methyl acrylate, methyl methacrylate, isobornyl acrylate, isobornyl methacrylate, isobutyl acrylate, isobutyl methacrylate, 2-ethylhexyl acrylate, 2-ethylhexyl methacrylate, dodecyl acrylate, dodecyl methacrylate, stearyl acrylate, stearyl methacrylate, trifluoroethyl acrylate and trifluoroethyl methacrylate.

Further non-limiting mention may also be made of acrylic acid, methacrylic acid, methacryloxypropyltris(trimethylsiloxy)silane, acryloxypropyltris(trimethylsiloxy)silane, acryloxypropylpolydimethylsiloxane and methacryloxypropylpolydimethylsiloxane.

Among the additional monomers that may be present in the block comprising the at least one monomer with an optical effect of formula (I), and/or that may be present in the at least one other block not comprising a monomer with an optical effect of formula (I), non-limiting mention may be made of the monomers with an optical effect of formulae (A), (B) and/or (C): 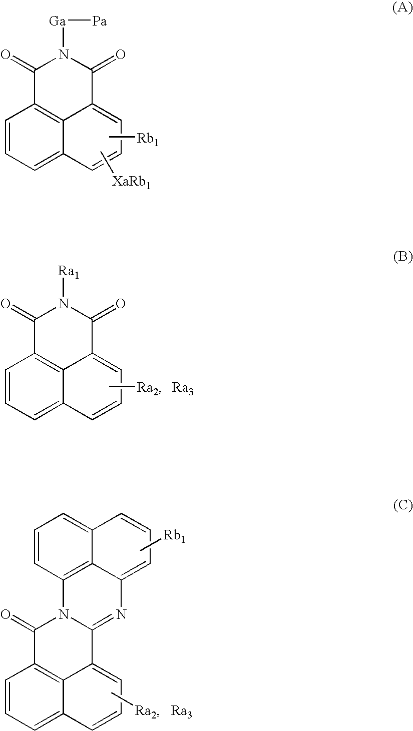
wherein:

Ra₁ is chosen from linear, branched and/or cyclic, saturated and/or unsaturated carbon-based radicals comprising from 1 to 32 carbon atoms, optionally substituted with at least one group chosen from ═O, OH, and NH₂ groups and halogen atoms, and/or optionally interrupted with at least one heteroatom chosen from O, N, P, Si and S atoms;

Rb₁ is chosen from a hydrogen atom; halogen atoms; linear, branched and/or cyclic, saturated and/or unsaturated carbon-based radicals comprising from 1 to 12 carbon atoms, optionally substituted with at least one group chosen from ═O, OH and NH₂ groups, and/or optionally interrupted with at least one heteroatom chosen from O, N, P, Si and S atoms; and groups NRR′ wherein R and R′, which may be identical or different, are chosen from a hydrogen atom and linear, cyclic or branched, saturated C₁₋₆ hydrocarbon-based radicals, such as methyl, ethyl, propyl, isopropyl, n-butyl, isobutyl, tert-butyl, pentyl or hexyl;

Ra₂ and Ra₃, which can be present on the same ring or each on a different ring, and which may be identical or different, are chosen from hydrogen atoms, halogen atoms, and groups of formula -Xa-Ga-Pa (II), with the proviso that at least one of the radicals Ra₂ and/or Ra₃ is a group of formula (II), in which:

Xa is chosen from oxygen and sulfur atoms, and —SO—, —SO₂—, —NH— and —NR₄— groups in which R₄ is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;

Ga is chosen from linear, branched and/or cyclic, saturated and/or unsaturated divalent carbon-based radicals comprising from 1 to 32 carbon atoms, optionally substituted with at least one entity chosen from ═O, OH, and NH₂ groups, and halogen atoms, and/or optionally interrupted with at least one heteroatom chosen from O, N, P, Si and S atoms;

Pa is a polymerizable group chosen from those of formulae (IIIa) to (IIIc): 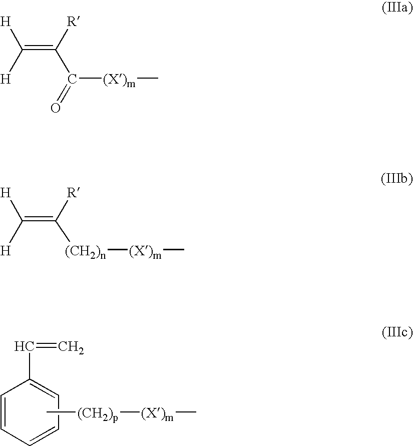
wherein:

R′ is chosen from a hydrogen atom and linear and branched, saturated C₁₋₆ hydrocarbon-based radicals,

X′ is chosen from an oxygen atom, and NH and NR″ groups, with R″ being chosen from C₁₋₆ alkyl, C₆₋₁₀aryl, (C₆₋₆)aryl(C₁₋₆)alkyl and (C₁₋₆)alkyl(C₆₋₁₀)aryl radicals aryl groups also possibly being substituted with at least one entity chosen from halogen atoms, and OH, C₁₋₆ alkoxy and C₆₋₁₀ aryloxy groups; and

m is equal to 0 or 1; n is equal to 0 or 1; p is equal to 0, 1 or 2.

For example, the at least one monomer with an optical effect of formula (I) may be present in an amount ranging from 0.01% to 100% by weight, such as from 0.1% to 99.99% by weight, for instance from 0.5% to 70% by weight and from 1% to 40% by weight, for example from 1.5% to 30% by weight, relative to the weight of the block comprising it (them).

As a further example, the at least one monomer with an optical effect of formula (I) may be present in an amount ranging from 0.01% to 70% by weight, such as from 0.1% to 50% by weight and from 0.5% to 30% by weight, for instance, from 1% to 20% by weight, relative to the total weight of the polymer.

Each of the blocks of the polymer according to the present disclosure may comprise at least one monomer of formula (I), which may be identical or different depending on the block.

The additional monomers may be present in an amount ranging from 0 to 99.99% by weight, for instance, from 0.01% to 99.9% by weight, such as from 30% to 99.5% by weight and from 60% to 99% by weight, for example from 70% to 98.5% by weight, relative to the weight of the block comprising them and comprising the at least one monomer with an optical effect of formula (I). The additional monomers are present in a proportion of 100% by weight in the possible block(s) not comprising any monomer with an optical effect of formula (I).

The additional monomers may be present in an amount ranging from 30% to 99.99% by weight, for instance from 50% to 99.9% by weight, such as from 70% to 99.5% by weight, and from 80% to 99% by weight, relative to the total weight of the polymer.

In general, for instance, the intermediate block (or segment) can comprise at least one constituent monomer m1 of the first block for example, chosen from the additional monomers, and at least one constituent monomer m2 of the second block chosen from the additional monomers other than the monomer m1.

In one embodiment of the present disclosure, the polymer may comprise a first block that comprises from 0.5% to 15% by weight, for example from 1% to 10% by weight of the at least one monomer of formula (I), and 85% to 99.5% by weight, such as from 90% to 99% by weight of additional monomers, relative to the total weight of the first block. The second block for instance, can comprise 100% by weight of additional monomers; the intermediate block (or segment) may comprise at least one constituent monomer m1 of the first block chosen from the additional monomers, and at least one constituent monomer m2 of the second block chosen from the additional monomers other than the monomer m1.

For example, in the polymer according to the present disclosure, the first block may be chosen from:

a) a block with a Tg of greater than or equal to 40° C.,

b) a block with a Tg of less than or equal to 20° C., and

c) a block with a Tg of between 20° C. and 40° C.,

and the second block may be chosen from a block of category a), b) and c) with the proviso that the second block is different from the first block.

The block comprising the at least one monomer with an optical effect of formula (I) can have, for example, a Tg of greater than or equal to 40° C., for instance greater than or equal to 60° C. In this case, the other block(s), if they do not comprise any monomers with an optical effect of formula (I), may have a Tg of less than or equal to 40° C., such as less than or equal to 20° C. If a second block comprises at least one monomer with an optical effect of formula (I), it may have, for instance, a Tg of less than or equal to 40° C., such as less than or equal to 20° C.

When the polymer comprises a block with a Tg of greater than or equal to 40° C., this block may have, for example, a Tg ranging from 40° C. to 150° C., for instance, from 50° C. to 120° C., such as from 60° C. to 120° C. It may then comprise, for example, in total or in part, monomers whose homopolymers have a Tg in the desired range, for instance greater than or equal to 40° C. It may also comprise monomers with a Tg outside this range. These monomers and their concentration can be chosen in an appropriate manner by a person skilled in the art, for instance on the basis of Fox's law, to obtain a block of desired Tg.

Among useful monomers, non-limiting mention may be made of:

the methacrylates of formula: CH₂═C(CH₃)—COOR₁

wherein R₁ is chosen from linear and branched unsubstituted alkyl groups comprising from 1 to 4 carbon atoms, such as a methyl, ethyl, propyl or isobutyl groups; and C₄ to C₁₂ cycloalkyl groups, such as isobornyl;

the acrylates of formula: CH₂═CH—COOR₂

wherein R₂ is chosen from tert-butyl groups and C₄ to C₁₂ cycloalkyl groups such as an isobornyl group;

the (meth)acrylamides of formula: CH₂═CR′—CO—NR₇R₈

wherein R′ is chosen from hydrogen atoms and CH₃ groups, and R₇ and R₈, which may be identical or different, are chosen from hydrogen atoms, and linear and branched C₁ to C₁₂ alkyl groups, such as n-butyl, t-butyl, isopropyl, isohexyl, isooctyl and isononyl groups; or alternatively R₇ is a hydrogen atom and R₈ is a 1,1-dimethyl-3-oxobutyl group,

and mixtures thereof.

Among the monomers whose homopolymers have a glass transition temperature Tg of greater than or equal to 40° C. that may be used as disclosed herein, non-limiting mention may be made of methyl methacrylate, ethyl methacrylate, isobutyl methacrylate, tert-butyl (meth)acrylate, (meth)acrylic acid, isobornyl (meth)acrylate, N-butylacrylamide, N-t-butylacrylamide, N-isopropylacrylamide, N,N-dimethylacrylamide and N,N-dibutylacrylamide, and mixtures thereof.

When the polymer comprises a block with a Tg of less than or equal to 20° C., this block may have, for example, a Tg ranging from −100° C. to 20° C., for instance from −80° C. to 15° C., such as from −50° C. to 0° C. The polymer may then comprise, for example, in total or in part, monomers whose homopolymers have a Tg in the desired range, for instance, less than or equal to 20° C. It may also comprise monomers with a Tg outside this range. These monomers and their concentration can be chosen in an appropriate manner by a person skilled in the art, for example, on the basis of Fox's law, to obtain a block of desired Tg.

Among the monomers with a Tg of less than or equal to 20° C., non-limiting mention may be made of:

the acrylates of formula CH₂═CHCOOR₃,

wherein R₃ is chosen from linear and branched, unsubstituted C₁ to C₁₂ alkyl groups, with the exception of the tert-butyl group, optionally intercalated with at least one heteroatom chosen from O, N and S atoms,

the methacrylates of formula CH₂═C(CH₃)—COOR₄,

wherein R₄ is chosen from linear and branched, unsubstituted C₆ to C₁₂ alkyl groups optionally intercalated with at least one heteroatom chosen from O, N and S atoms;

the vinyl esters of formula R₅—CO—O—CH═CH₂

wherein R₅ is chosen from linear and branched C₄ to C₁₂ alkyl groups;

C₄-C₁₂ alkyl vinyl ethers, such as methyl vinyl ether and ethyl vinyl ether;

N—(C₄ to C₁₂ alkyl) acrylamides such as N-octylacrylamide,

and mixtures thereof.

Among these monomers, further non-limiting mention may be made of, for example, methyl acrylate, ethyl acrylate, isobutyl acrylate and 2-ethylhexyl (meth)acrylate, and mixtures thereof.

When the polymer comprises a block with a Tg of between 20° C. and 40° C., it may comprise, for example, in total or in part, monomers whose homopolymers have a Tg in the desired range, and among which non-limiting mention may be made of n-butyl methacrylate, cyclohexyl acrylate, cyclododecyl acrylate, neopentyt-acrylate and isodecylacrylamide, and mixtures thereof.

In one embodiment of the present disclosure, the polymer according to the present disclosure comprises in at least one block, for example, in each of the blocks, at least one monomer chosen from (meth)acrylic acid esters; it may optionally also comprise at least one second monomer chosen from acrylic acid and methacrylic acid, and mixtures thereof.

For instance, all the monomers other than the monomers with optical effects of formula (I) may be chosen from (meth)acrylic acid esters and (meth)acrylic acid.

According to another embodiment of the present disclosure, the polymer as disclosed herein comprises at least one first block with a Tg of greater than or equal to 40° C., for instance greater than or equal to 50° C., such as greater than or equal to 60° C., and at least one second block with a Tg of less than or equal to 20° C., for instance less than or equal to 10° C., such as less than or equal to 0° C.

For example, in the final polymer, the block with a Tg of greater than or equal to 40° C., may be present in an amount ranging from 20% to 95% by weight, for instance from 30% to 80%, such as from 50% to 75% by weight, relative to the weight of the final polymer. For example, in the final polymer, the block with a Tg of less than or equal to 20° C. may be present in an amount.ranging from 5% to 80% by weight, for instance from 15% to 50%, such as from 25% to 45% by weight, relative to the weight of the final polymer.

The weight-average mass (Mw) of the polymer according to the present disclosure can be, for instance, less than or equal to 300,000; it can range, for example, from 35,000 to 200,000, such as from 40,000 to 150,000.

The number-average mass (Mn) of the polymer according to the present disclosure can be, for instance, less than or equal to 70,000; it can range, for example, from 5,000 to 60,000, such as from 6,000 to 50,000.

The weight-average (Mw) and number-average (Mn) molar masses are determined by gel permeation liquid chromatography (THF solvent, calibration curve established with linear polystyrene standards, refractometric and UV detector).

For example, the polydispersity index of the polymer according to the present disclosure can be greater than 2, for example ranging from 2 to 9, such as greater than or equal to 2.5, for example ranging from 2.5 to 8, for instance greater than or equal to 2.8 and ranging from 2.8 to 7. The polydispersity index Ip of the polymer is equal to the ratio of the weight-average mass Mw to the number-average mass Mn.

The polymer according to the present disclosure can have, for example, an absorption wavelength ranging from 200 nm to 550 nm, for instance from 220 nm to 520 nm, such as from 240 nm to 500 nm.

The polymer can have, for example, an emission wavelength ranging from 350 nm to 750 nm, for instance ranging from 390 nm to 700 nm, such as from 420 nm to 670 nm.

The polymer according to the present disclosure can be obtained by solution free-radical polymerization according to the following preparation process:

a portion of the polymerization solvent can be introduced into a suitable reactor, and the system is heated until the appropriate temperature for the polymerization (typically ranging from 60° C. and 120° C.) is reached,

once this temperature has been reached, the constituent monomers of the first block may be added, in the presence of some of the polymerization initiator,

after a period of time T corresponding to a maximum degree of conversion, for instance, of 90%, the constituent monomers of the second block and the rest of the initiator can be introduced,

the mixture is left to react for a period of time T′ (ranging, for example, from 3 to 6 hours), at the end of which the mixture is cooled to room temperature (25° C.) so as to obtain the polymer dissolved in the polymerization solvent.

As used herein, the term “polymerization solvent” is understood to mean a solvent or a solvent mixture chosen for instance, from ethyl acetate, butyl acetate, C₁-C₆ alcohols such as isopropanol or ethanol, aliphatic alkanes such as isododecane, and mixtures thereof. For example, the polymerization solvent may be a mixture of butyl acetate and isopropanol, or isododecane.

For example, the polymer according to the present disclose can be not water-soluble, i.e., the polymer is not soluble in water or in a mixture of water and of linear or branched monoalcohols comprising from 2 to 5 carbon atoms, such as ethanol, isopropanol or n-propanol, without a pH modification, when present in an active material amount of at least 1% by weight, at room temperature (25° C.).

The present disclosure also relates to compositions, such as cosmetic compositions, comprising at least one polymer of specific structure, as described above, in a physiologically acceptable, such as a cosmetically acceptable medium.

The polymers according to the present disclosure may be present, alone or as a mixture, in the compositions according to the present disclosure in an amount ranging from 0.01% to 75% by weight, for instance from 0.1% to 70% by weight, such as from 1% to 65% by weight, from 3% to 60% by weight, from 5% to 50% by weight, and from 6% to 25% by weight, relative to the total weight of the composition.

The polymers can be present in the composition in dissolved form, for example in water, in an oil or in an organic solvent, or alternatively in the form of an aqueous or organic dispersion.

For example, the polymers according to the present disclosure may be soluble or dispersible in at least one of the phases of the composition comprising them.

The cosmetic or pharmaceutical compositions according to the present disclosure comprise, besides the polymers as disclosed herein, a physiologically acceptable medium, for instance a cosmetically, dermatologically or pharmaceutically acceptable medium, i.e., a medium that is compatible with keratin materials such as facial or bodily skin, the hair, the eyelashes, the eyebrows and the nails.

The composition can thus comprise a hydrophilic medium comprising water or a mixture of water and at least one hydrophilic organic solvent, for instance alcohols such as linear or branched lower monoalcohols comprising from 2 to 5 carbon atoms, for instance ethanol, isopropanol or n-propanol, and polyols, for instance glycerol, diglycerol, propylene glycol, sorbitol or pentylene glycol, and polyethylene glycols, or alternatively hydrophilic C₂ ethers and C₂-C₄ aldehydes.

The water or the mixture of water and at least one hydrophilic organic solvent can be present in the composition according to the present disclosure in an amount ranging from 0.1% to 99% by weight, such as from 10% to 80% by weight, relative to the total weight of the composition.

The composition can also be anhydrous.

The composition can also comprise a fatty phase which can comprise fatty substances that are liquid at room temperature (25° C.) and/or of fatty substances that are solid at room temperature, such as waxes, pasty fatty substances and gums, and mixtures thereof. These fatty substances may be of animal, plant, mineral or synthetic origin. This fatty phase may also comprise lipophilic organic solvents.

Among the fatty substances that are liquid at room temperature, often referred to as oils, which may be used according to the present disclosure, non-limiting mention may be made of: hydrocarbon-based oils of animal origin such as perhydrosqualene; hydrocarbon-based plant oils such as liquid triglycerides of fatty acids of 4 to 10 carbon atoms, for instance heptanoic or octanoic acid triglycerides, or alternatively sunflower oil, maize oil, soybean oil, grapeseed oil, sesame seed oil, apricot oil, macadamia oil, castor oil, avocado oil, caprylic/capric acid triglycerides, jojoba oil, shea butter, linear or branched hydrocarbons of mineral or synthetic origin, such as liquid paraffin and derivatives thereof, petroleum jelly, polydecenes, hydrogenated polyisobutene such as parleam; synthetic esters and ethers, for example, of fatty acids, for instance purcellin oil, isopropyl myristate, 2-ethylhexyl palmitate, 2-octyldodecyl stearate, 2-octyldodecyl erucate, isostearyl isostearate; hydroxylated esters, for instance isostearyl lactate, octyl hydroxystearate, octyldodecyl hydroxystearate, diisostearyl malate, triisocetyl citrate, and fatty alcohol heptanoates, octanoates and decanoates; polyol esters, for instance propylene glycol dioctanoate, neopentyl glycol diheptanoate and diethylene glycol diisononanoate; and, pentaerythritol esters; fatty alcohols comprising from 12 to 26 carbon atoms, for instance octyldodecanol, 2-butyloctanol, 2-hexyldecanol, 2-undecylpentadecanol and oleyl alcohol; partially hydrocarbon-based fluoro oils and/or partially silicone-based fluoro oils; silicone oils, for instance volatile or non-volatile, linear or cyclic polymethylsiloxanes (PDMSs), which are liquid or pasty at room temperature, for instance cyclomethicones, dimethicones, optionally comprising a phenyl group, for instance phenyl trimethicones, phenyltrimethylsiloxydiphenylsiloxanes, diphenylmethyidimethyltrisiloxanes, diphenyl dimethicones, phenyl dimethicones and polymethylphenylsiloxanes; and mixtures thereof.

These oils may be present in an amount ranging from 0.01% to 90%, such as from 0.1% to 85% by weight, relative to the total weight of the composition.

The composition according to the present disclosure may also comprise at least one physiologically acceptable organic solvent.

The at least one organic solvent can be present in an amount ranging from 0.1% to 90%, for instance from 0.5% to 85%, such as from 10% to 80% and from 30% to 50% by weight, relative to the total weight of the composition.

Among the organic solvents that may be used, non-limiting mention may be made for instance, besides the hydrophilic organic solvents mentioned above, of ketones that are liquid at.room temperature such as methyl ethyl ketone, methyl isobutyl ketone, diisobutyl ketone, isophorone, cyclohexanone and acetone; propylene glycol ethers that are liquid at room temperature, such as propylene glycol monomethyl ether, propylene glycol monomethyl ether acetate, and dipropylene glycol mono-n-butyl ether; short-chain esters (comprising from 3 to 8 carbon atoms in total), such as ethyl acetate, methyl acetate, propyl acetate, n-butyl acetate and isopentyl acetate; ethers that are liquid at 25° C., such as diethyl ether, dimethyl ether or dichlorodiethyl ether; alkanes that are liquid at 25° C., such as decane, heptane, dodecane, isododecane and cyclohexane; aromatic cyclic compounds that are liquid at 25° C., such as toluene and xylene; aldehydes that are liquid at 25° C., such as benzaldehyde and acetaldehyde, and mixtures thereof.

As used herein, the term “wax” is understood to mean a lipophilic compound that is solid at room temperature (25° C.), which can undergo a reversible solid/liquid change of state, and which has a melting point of greater than or equal to 25° C., which may be up to 120° C. By bringing the wax to the liquid state (melting), it is possible to make it miscible with the oils possibly present and to form a microscopically homogeneous mixture, but, upon returning the temperature of the mixture to room temperature, recrystallization of the wax is obtained in the oils of the mixture. The melting point of the wax may be measured using a differential scanning calorimeter (DSC), for example the calorimeter sold under the name DSC 30 by the company Mettler.

The waxes can be hydrocarbon-based waxes, fluoro waxes and/or silicone waxes and can be of plant, mineral, animal and/or synthetic origin. For instance, the waxes can have a melting point of greater than 30° C., such as greater than 45° C. Among waxes that may be used in the composition of the present disclosure, non-limiting mention may be made of beeswax, carnauba wax or candellila wax, paraffin, microcrystalline waxes, ceresin or ozokerite, synthetic waxes, for instance polyethylene waxes or Fischer-Tropsch waxes, and silicone waxes, for instance alkyl or alkoxy dimethicones comprising from 16 to 45 carbon atoms.

The gums can be polydimethylsiloxanes (PDMSs) of high molecular weight, and cellulose gums or polysaccharides, and the pasty substances are generally hydrocarbon-based compounds, for instance lanolins and derivatives thereof, or PDMSs.

The nature and amount of the solid substances used depend on the desired mechanical properties and textures. As a guide, the at least one wax can be present in the composition in an amount ranging from 0.1% to 50% by weight, such as from 1% to 30% by weight, relative to the total weight of the composition.

The composition according to the present disclosure can also comprise, in a particulate phase, at least one pigment and/or nacre and/or filler usually used in cosmetic compositions.

The composition can also comprise at least one dyestuff chosen from water-soluble dyes and/or liposoluble dyes that are well known to those skilled in the art.

As used herein, the term “pigments” is understood to mean white or colored, mineral or organic particles of any shape, which are insoluble in the physiological medium and which are intended to color the composition.

As used herein, the term “fillers” is understood to mean colorless or white, mineral or synthetic, lamellar or non-lamellar particles intended to give body or rigidity to the composition, and/or softness, and/or a matt effect and/or uniformity to the makeup result.

As used herein, the term “nacres” is understood to mean iridescent particles of any form, produced for example, by certain molluscs in their shell, or else synthesized.

The at least one pigment may be present in the composition in an amount ranging from 0.01% to 25%, for instance from 3% to 10% by weight, relative to the total weight of the composition. The pigments may be white or colored, and mineral or organic. Mention may be made, as non-limiting examples, of titanium oxide, zirconium oxide or cerium oxide, and also zinc oxide, iron oxide or chromium oxide, ferric blue, chromium hydrate, carbon black, ultramarines (aluminosilicate polysulfides), manganese pyrophosphate and certain metallic powders such as silver or aluminium powder. Non-limiting mention may also be made of the D&C pigments and lakes commonly used to give the lips and the skin a makeup effect, which are calcium, barium, aluminium, strontium or zirconium salts.

The at least one nacre may be present in the composition in an amount ranging from 0.01% to 20% by weight, for instance ranging from 3% to 10% by weight, relative to the total weight of the composition. Among the nacres that may be envisaged for use as disclosed herein, non-limiting mention may be made of natural mother-of-pearl, mica coated with titanium oxide, with iron oxide, with natural pigment or with bismuth oxychloride, and also colored titanium mica.

The at least one liposoluble and/or water-soluble dye may be present in an amount ranging from 0.001% to 15% by weight, for instance, from 0.01% to 5% by weight, such as from 0.1% to 2% by weight, relative to the total weight of the composition. Among the at least one liposoluble and/or water-soluble dye that may be present in the composition, alone or as a mixture, non-limiting mention may be made of the disodium salt of ponceau, the disodium salt of alizarin green, quinoline yellow, the trisodium salt of amaranth, the disodium salt of tartrazine, the monosodium salt of rhodamine, the disodium salt of fuchsin, xanthophyll, methylene blue, cochineal carmine, halo-acid dyes, azo dyes, anthraquinone dyes, copper sulfate, iron sulfate, Sudan brown, Sudan red and annatto, and also beetroot juice and carotene.

The composition according to the present disclosure may also comprise at least one filler, which can be present, for example, in an amount ranging from 0.01% to 50% by weight, such as from 0.02% to 30% by weight, relative to the total weight of the composition. The fillers may be mineral or organic in any form, platelet-shaped, spherical or oblong. Mention may be made, by way of non-limiting example, of talc, mica, silica, kaolin, polyamide (Nylon®) powders, poly-o-alanine powder and polyethylene powder, powders of tetrafluoroethylene polymers (Teflon@), lauroyllysine, starch, boron nitride, hollow polymer microspheres such as those of polyvinylidene chloride/acrylonitrile, for instance Expancel® (Nobel Industrie) or acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, such as from 12 to 18 carbon atoms; for example zinc, magnesium or lithium stearate, zinc laurate or magnesium myristate.

The composition may also comprise at least one additional polymer such as a-film-forming polymer. As disclosed herein, the term “film-forming polymer” is understood to mean a polymer capable, by itself or in the presence of an auxiliary film-forming agent, of forming a continuous film that adheres to a support, for instance, to keratin materials. Among the film-forming polymers that may be used in the composition of the present disclosure, non-limiting mention may be made of synthetic polymers, of free-radical type or of polycondensate type, polymers of natural origin and mixtures thereof, for example acrylic polymers, polyurethanes, polyesters, polyamides, polyureas and cellulose-based polymers, for instance nitrocellulose.

The composition according to the present disclosure may also comprise at least one ingredient commonly used in cosmetics, such as vitamins, thickeners, gelling agents trace elements, softeners, sequestering agents, fragrances, acidifying or basifying agents, preserving agents, sunscreens, surfactants, antioxidants, agents for preventing hair loss, antidandruff agents, propellants and ceramides or mixtures thereof.

Needless to say, a person skilled in the art will take care to select this or these optional additional compound(s), and/or the amount thereof, such that the beneficial properties of the composition according to the present disclosure are not, or are not substantially, adversely affected by the envisaged addition.

The composition according to the present disclosure may be in the form of a suspension, a dispersion, for instance of oil in water by means of vesicles; an optionally thickened or even gelled aqueous or oily solution; an oil-in-water, water-in-oil or multiple emulsion; a gel or a mousse; an oily or emulsified gel; a dispersion of vesicles, such as lipid vesicles; a two-phase or multiphase lotion; a spray; a free, compact or cast powder; an anhydrous paste. This composition may have the appearance of a lotion, a cream, a salve, a soft paste, an ointment, a mousse, a cast or molded solid, for example in stick or dish form, or a compacted solid.

A person skilled in the art will be able to choose the appropriate form for the composition, and also the method for preparing it, on the basis of his general knowledge, taking into account the nature of the constituents used, such as their solubility in the support, and the intended application of the composition.

The cosmetic composition according to the present disclosure can be in the form of a care and/or makeup product for bodily or facial skin, the lips, the nails, the eyelashes, the eyebrows and/or the hair, an antisun product, a self-tanning product, or a hair product for caring for, treating, shaping, making up or coloring the hair. It can also be in the form of a makeup composition, for instance a complexion product such as a foundation, a makeup rouge or an eyeshadow; a lip product such as a lipstick or a lipcare product; a concealer product; a blusher, a mascara or an eyeliner; an eyebrow makeup product, a lip pencil or an eye pencil; a nail product such as a nail varnish or a nailcare product; a body makeup product; a hair makeup product (hair mascara or hair lacquer).

The composition can also be in the form of a protective or care composition for the skin of the face, the neck, the hands or the body, for example an anti-wrinkle composition, a moisturizing or treating composition; an antisun composition or an artificial tanning composition. The composition can also be in the form of a hair product, for instance for coloring, holding the hairstyle, shaping the hair, caring for, treating or cleansing the hair, such as shampoos, hairsetting gels or lotions, blow-drying lotions, and fixing and styling compositions such as lacquers or sprays.

In one embodiment of the present disclosure, the composition can be in the form of a nail varnish that comprising, in addition to the at least one polymer according to the present disclosure, at least one organic solvent, at least one film-forming polymer, and optionally at least one pigment and/or at least one dye.

In another embodiment of the present disclosure, the composition can be in the form of a foundation that may comprise, in addition to the at least one poymer according to the present disclosure, at least one oil in a fatty phase, at least one pigment, and optionally an aqueous phase.

In yet another embodiment of the present disclosure, the composition can be in the form of an anti-ageing or anti-wrinkle composition, for instance intended to be applied to the face and/or the neck, such as to the wrinkled areas of the face, for example around the eyes. For instance, it has been found, surprisingly, that the polymers according to the present disclosure makes it possible to obtain a composition that may be applied to the skin and that can give an immediate tensioning effect on already-formed wrinkles and/or fine lines; the use of these polymers as tensioning agents can be beneficial, as they can make it possible to form an effective tensioning film with effective rigidity, while at the same time being supple so as to avoid an annoying tautness of keratin materials such as the skin, during the application of a composition comprising such agents. In this case, the block polymer can be, for example, non-elastomeric and water-insoluble. As used herein, the term “water-insoluble polymer” is understood to mean that the polymer is not soluble in water, or in a mixture of water and of linear or branched C₂-C₅ monoalcohols, for instance ethanol, isopropanol or n-propanol, without pH modification, when the active material is present in an amount of at least 1% by weight, at room temperature (25° C.).

In this embodiment, the first block can have, for instance, a Tg of greater than or equal to 85° C., for example ranging from 90° C. to 150° C., such as from 100° C. to 120° C. For example, the block with a Tg of greater than or equal to 85° C. can be present in an amount ranging from 50% to 90% by weight, relative to the weight of the final polymer, such as from 60% to 80% by weight of the final polymer. The second block can have, for example, a Tg of less than or equal to 20° C., for example ranging from −100° C. to 20° C., such as from −80° C. to 15° C. and from −70° C. to 10° C. For instance, the block with a Tg of less than or equal to 20° C. can be present in an amount ranging from 5% to 50% by weight, relative to the weight of the final polymer such as from 10% to 40% by weight, relative to the weight of the final polymer.

The composition according to the present disclosure may also comprise at least one anti-ageing active agent chosen for example, from desquamating agents, moisturizers, agents for stimulating keratinocyte proliferation and/or differentiation, agents for stimulating collagen and/or elastin synthesis or for preventing their degradation, depigmenting agents, anti-glycation agents, agents for stimulating glycoaminoglycan synthesis, dermo-decontracting agents or muscle relaxants, antioxidants and free-radical scavengers, and mixtures thereof.

Another aspect of the present disclosure is the use of the polymers as disclosed herein as tensioning agents in a cosmetic composition, such as in an anti-wrinkle composition.

Still another aspect of the present disclosure is a cosmetic process for treating wrinkled skin, such as the contour of the eyes, comprising applying to the skin a cosmetic composition comprising, in a cosmetically acceptable medium, at least one polymer as defined above.

Yet another aspect of the present disclosure is a cosmetic treatment process for instance, for making up or caring for keratin materials, such as bodily or facial skin, the lips, the nails; the eyelashes, the eyebrows and/or the hair, comprising the application to the keratin materials of a cosmetic composition as defined above.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contains certain errors necessarily resulting from the standard deviation found in their respective testing measurements.

The following examples are intended to illustrate the invention in a non-limiting manner.

### EXAMPLES

Method for Measuring the Wavelength (Emission and Absorption)

The wavelength measurement was performed using a Varian Cary Eclipse fluorimeter. Unless otherwise indicated, this measurement was performed in the following manner:

20 mg of product were placed in a 50 ml cylinder. To dissolve the product, the cylinder was filled to 50 ml with a suitable solvent, for example dichloromethane (DCM), chloroform, isododecane, heptane or dimethyl sulfoxide (DMSO). The resulting solution was mixed and 250 microlitres were taken and placed in a 50 ml cylinder, which was then filled to 50 ml again with the solvent.

The whole was mixed and a sample of the solution was taken and placed in a closed quartz cuvette 10 mm thick, which was then placed in the measuring chamber.

### Example 1

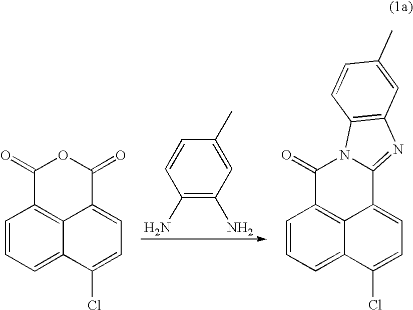

179.9 g (0.77 mol) of 4-chloro-1,8-naphthalic anhydride were placed in a 1 litre three-necked flask, under an inert atmosphere (nitrogen), followed by addition of 1 litre of 2-(2-ethoxyethoxy)ethanol. The mixture was heated to 100° C. and turned a clear orange color. 100 g (0.78 mol) of 3,4-diaminotoluene predissolved in 300 ml of 2-(2-ethoxyethoxy)ethanol were added dropwise, with stirring. The dropping funnel was rinsed with 200 ml of this solvent. The reaction solution was heated at 150° C. for 18 hours. The resulting reaction mixture was then allowed to cool to room temperature. The precipitate was filtered off on a sinter funnel and then washed with ethanol. It was recovered and dried under vacuum. 211.3 g of orange crystals are obtained (72.6% yield).

Characterization

¹H-NMR (CDCl₃, 400 MHz) δ: 8.74-8.72 (1H), 8.61-8.42 (3H), 7.83-7.56 (3H), 7.24-7.22 (1H), 2.54-2.51 (3H).

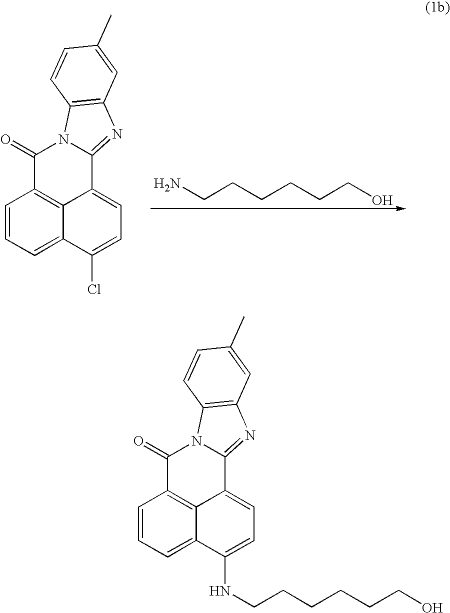

13.6 g (42.7 mmol) of p-tolyl-4-chloroisoquinolinone were placed in a three-necked round-bottomed flask equipped with a Dimroth condenser and placed under an inert atmosphere of argon, and 100.0 g (0.85 mol) of 6-amino-1-hexanol were added. The mixture was stirred and heated to 180° C. in order to obtain a homogeneous mixture; the resulting mixture was left to react for 16 hours and was then allowed to cool to room temperature, and a red precipitate was formed. The precipitate was filtered off and washed with ethanol. 17.0 g of desired product were obtained.

Characterization

¹H-NMR (DMSO, 400 MHz) δ: 8.82-8.20 (4H), 7.96-7.49 (3H), 7.28-7.15(1H), 6.82-6.80 (1H), 4.37 (1H), 3.41-3.39 (2H), 2.49-2.45 (5H), 1.72-1.69 (2H), 1.48-1.36 (6H).

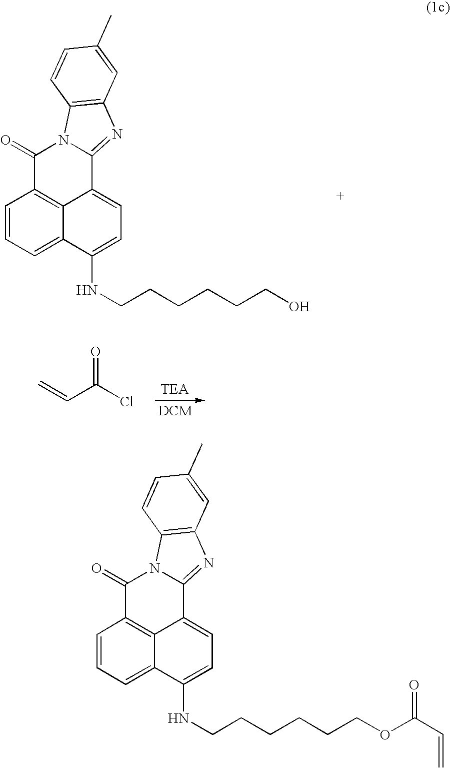

5.0 g (12.5 mmol) of p-tolyl-4-(6-hexanol)isoquinolinone were placed in a three-necked round-bottomed flask equipped with a Dimroth condenser and placed under an inert atmosphere of argon. 150 ml of dry dichloromethane were added and the mixture was stirred until a homogeneous solution was obtained. 7.0 ml (50 mmol) of triethanolamine-and then 4.1 ml (50 mmol) of acryloyl chloride were then added, with stirring at 0° C. The temperature was allowed to rise to 25° C. The reaction progress was monitored by TLC and, when it was observed that everything has reacted, 50 ml of water were added. The reaction solution was then washed with saline water+sodium bicarbonate (brine) and then again with water, and dried over sodium sulfate. The solvents were evaporated off under reduced pressure to give 4.1 g (73.5% yield) of a red powder.

Characterization

¹H-NMR (DMSO, 500 MHz) δ: 8.70-8.68 (1H), 8.63-8.60 (1H), 8.44-8.42 (1H), 8.33-8.29 (1H), 7.90-7.88 (1H), 7.73-7.61 (2H), 7.27-7.23 (1H), 6.82-6.81 (1H), 6.32-6.29 (1H), 6.18-6.12 (1H), 5.92-5.89 (1H), 4.13-4.11 (2H), 3.41-3.37 (2H), 1.74-1.62 (4H), 1.47-1.40 (4H).

λₘₐₓabsorption: 377 nm

λₘₐₓemission: 556 nm (red)

### Example 2

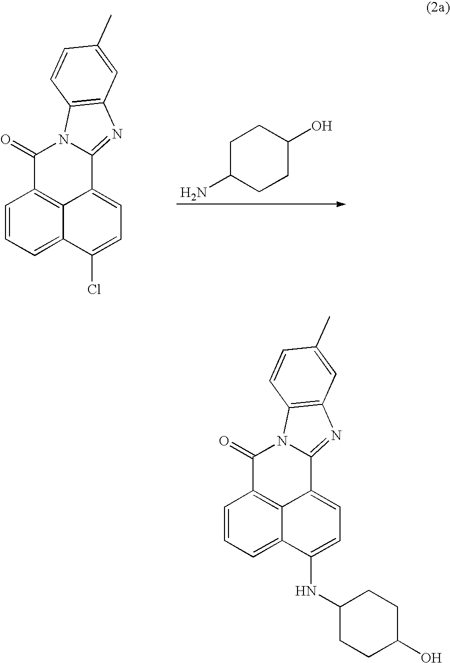

8.0 g (25.1 mmol) of p-tolyl-4-chloroisoquinolinone and 8.7 g of trans-aminocyclohexanol (75.3 mmol, 3 eq) were placed in a microwave reactor. 30 ml of NMP were added. The reactor was placed in the microwave tank with a glass paddle stirrer. The mixture-was heated to 130° C. with stirring over 5 minutes and was then maintained at 130° C. for 4 hours. The solid medium gradually became liquid and changed from a yellow-green pasty solution to a Bordeaux-red liquid solution. A TLC was performed (9/1 CH₂Cl₂/MeOH) to determine the end of the reaction. The reaction solution was then poured into 600 ml of a sodium hydrogen carbonate solution; the product precipitates out immediately; it was washed three times with 600 ml of water and then dried under vacuum to give 9.07 g of bright Bordeaux-red powder (90.9% yield).

Characterization

¹H-NMR (DMSO, 400 MHz) δ: 8.89-8.65 (2H), 8.50-8.42 (1H), 8.34-8.23 (1H), 7.76-7.18 (4H), 6.94-6.91 (1H), 4.65 (1H), 3.66-3.63 (1H), 3.52-3.47 (1H), 2.51-2.47 (3H), 2.09-2.02 (2H), 1.94-1.92 (2H), 1.55-1.37 (4H).

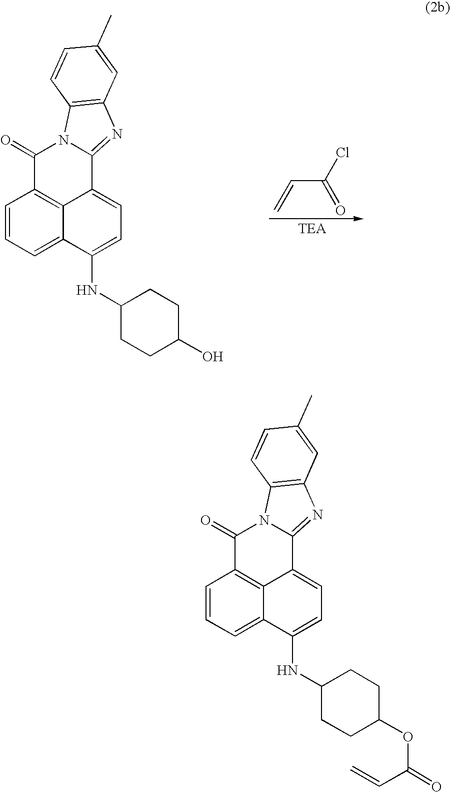

10.0 g (25 mmol) of p-tolyl-4-(4-hydroxycyclohexylamino)isoquinolinone were placed in a 1 litre three-necked flask under an inert atmosphere (argon), followed by addition of 350 ml of dichloromethane. The mixture was stirred until a homogeneous solution was obtained. 3.31 g (33 mmol) of triethanolamine were then added. 3.0 g (33 mmol) of acryloyl chloride in 50 ml of dichloromethane were then added dropwise, with stirring (500 rpm) at 5° C., and the reaction solution was then heated to 40° C. The reaction was monitored by TLC until the starting material had disappeared. After reaction for 3 hours, the organic phase was washed with water and with sodium bicarbonate, and again with water. The organic phase was dried over sodium sulfate and the solvents were evaporated off. The crude product obtained was purified on silica. 5.1 g of the desired product were recovered (45.2% yield).

Characterization

¹H-NMR (DMSO, 400 MHz) δ: 8.89-8.65 (2H), 8.50-8.42 (1H), 8.34-8.23 (1H), 7.76-7.18 (4H), 6.94-6.91 (1H), 6.37-6.32 (1H), 6.23-6.16 (1H), 5.96-5.94 (1H), 4.71-4.76 (1H), 3.75-3.74 (1H), 2.49-2.45 (3H), 2.11-2.05 (4H), 1.68-1.58 (4H).

### Example 3

33 g of isododecane were placed in a 500 ml reactor and then heated to 90° C.

2 g of the monomer prepared according to Example 1 were dissolved in 10 ml of toluene, and 40 g of isobornyl acrylate, 28 g of isobutyl methacrylate, 37 g of isododecane and 0.6 g of initiator 2,5-bis(2-ethylhexanoylperoxy)-2,5-dimethylhexane (Trigonox® 141 from Akzo Nobel) were then added. This mixture was stirred for 1 hour in the reactor at 90° C. The whole was maintained at 90° C. for 1 hour 15 minutes.

30 g of 2-ethylhexyl acrylate, 30 g of isododecane and 0.4 g of 2,5-bis(2-ethylhexanoylperoxy)-2,5-dimethylhexane were then introduced into the above mixture, at 90° C. and over 30 minutes. The mixture was maintained at 90° C. for 4 hours and was then cooled to 25° C.

After replacing the toluene with isododecane, a solution comprising 50% of polymer solids in isododecane is obtained.

This polymer comprises a first block of isobornyl acrylate, isobutyl methacrylate and optical-brightening monomer of formula (I), and a 2-ethylhexyl acrylate second block, and also an intermediate segment.

### Example 4

50 g of ethyl acetate were placed in a 500 ml reactor and then heated at 78° C. for 1 hour. 27.5 g of methyl methacrylate, 5 g of acrylic acid, 2.5 g of monomer prepared in Example 2 were dissolved in 20 g of THF, and 0.3 g of 2,5-bis(2-ethylhexanoylperoxy)-2,5-dimethylhexane (Trigonox® 141 from Akzo Nobel) were then added, at 78° C. and over 35 minutes. The mixture was maintained at 78° C. for 1 hour. 15 g of methyl acrylate and 0.2 g of 2,5-bis(2-ethylhexanoylperoxy)-2,5-dimethylhexane were then added at 78° C. and over 30 minutes. The mixture was maintained at 78° C. for 5 hours and then diluted with 75 g of butyl acetate. The ethyl acetate and the THF were distilled off under reduced pressure, 50 g of butyl acetate were then added and 50 g of butyl acetate were distilled off.

A solution comprising 43.1% of polymer solids in butyl acetate was obtained.

This polymer comprises a first block of methyl methacrylate, acrylic acid and monomer according to the present disclosure, and a methyl acrylate second block, and also an intermediate segment.

### Example 5

An anhydrous foundation comprising the following (weight %) was prepared:

| Component | Amount |
|---|---|
| polyethylene wax | 12% |
| volatile silicone oils | 25% |
| phenyl trimethicone | 20% |
| polymethyl methacrylate microspheres | 12% |
| polymer of Example 3 | 6% |
| isododecane | qs 100% |

Preparation:

The waxes were melted and, when it was all clear, the phenyl trimethicone and the silicone oils were added with stirring; the microspheres, the isododecane and the polymer were then added. The mixture was homogenized for 15 minutes and the resulting composition was then cast and allowed to cool.

An anhydrous foundation was obtained.

### Example 6

A nail varnish was prepared, comprising:

20% by weight of polymer according to Example 4

qs 100% organic solvents (butyl acetate and ethyl acetate).

### Example 7

A stick of lipstick was prepared, comprising:

| Component | Amount |
|---|---|
| polyethylene wax | 15% |
| polymer of Example 3 | 10% AM |
| hydrogenated polyisobutene (Parléam from Nippon Oil | 25% |
| Fats) | |
| pigments | 10% |
| isododecane | qs 100% |

1. A block polymer comprising at least one first block and at least one second block that are mutually incompatible,
wherein the at least one first and the at least one second blocks are linked together via an intermediate segment comprising at least one constituent monomer of the at least one first block and at least one constituent monomer of the at least one second block, and
wherein at least one of the blocks comprises at least one monomer of formula (I): 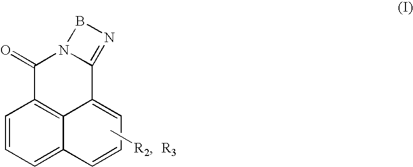
wherein:
R₂ and R₃, which are present on the same ring or each on a different ring, are chosen, independently, from hydrogen atoms, halogen atoms, and groups of formula (II): —X-G-P, with the proviso that at least one of the radicals R₂ and/or R₃ is a group of formula (II), in which:
X is chosen from oxygen and sulfur atoms, and —SO—, —SO₂—, —NH— and —NR— radicals, wherein R is a linear, branched and/or cyclic, saturated and/or unsaturated carbon-based radical comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
P is a polymerizable group chosen from those of formulae (IIIa) to (IIIc): 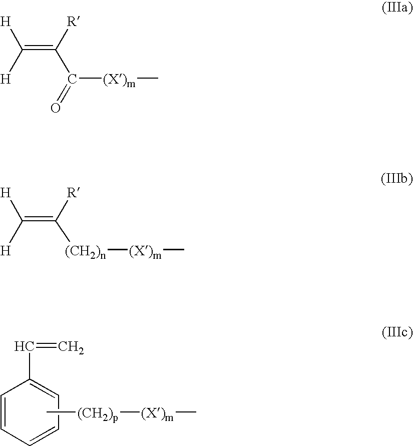
wherein:
R′ is chosen from a hydrogen atom and linear and branched, saturated C₁₋₆ hydrocarbon-based radicals,
X′ is chosen from an oxygen atom, and NH and NR″ groups, wherein R″ is chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, the alkyl and/or aryl radicals possibly being substituted with at least one entity chosen from halogen atoms, and OH, C₁₋₆ alkoxy and C₆₋₁₀ aryloxy groups;
m is equal to 0 or 1; n is equal to 0 or 1; p is equal to 0, 1 or 2;
B is a divalent aromatic group chosen from those of formulae (IVa) to (IVd): 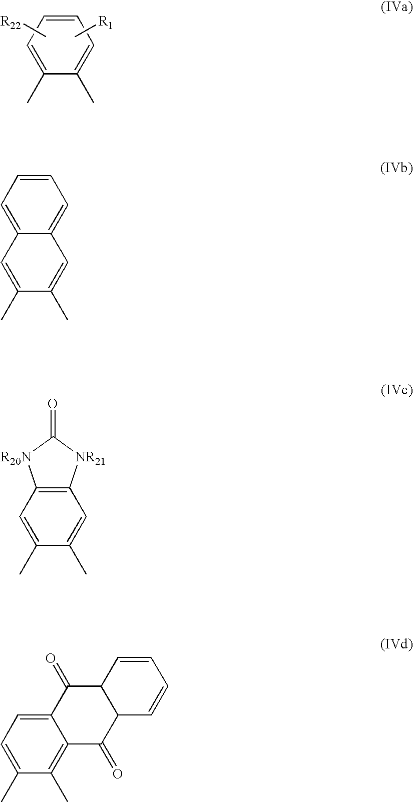
wherein:
R₁ is chosen from linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si, and S atoms;
R₂₂ is chosen from a hydrogen atom, and linear, branched and/or cyclic, saturated and/or unsaturated carbon-based radicals comprising from 1 to 32 carbon atoms, optionally substituted with at least one entity chosen from ═O, OH, and NH₂ groups, and halogen atoms, and/or optionally interrupted with at least one heteroatom chosen from O, N, P, Si, and S atoms;
R₂₀ and R₂₁, which may be identical or different, are chosen from hydrogen atoms, linear and branched C₁₋₈ alkyl radicals, and cyclopentyl, cyclohexyl, cyclooctyl, cyclodecyl, cyclododecyl, benzyl, naphthyl and phenyl radicals.

2. The block polymer according to claim 1, wherein R₂ is a hydrogen atom and R₃ is a group of formula (II).

3. The block polymer according to claim 1, wherein X is chosen from an oxygen atom, and —NH— and —NR— radicals, wherein R is chosen from linear, branched and/or cyclic, saturated and unsaturated hydrocarbon-based radicals optionally comprising a hydrocarbon-based ring that is itself saturated or unsaturated, comprising from 2 to 18 carbon atoms, optionally substituted with at least one entity chosen from ═O, OH, and NH₂ groups and halogen atoms, and/or optionally interrupted with at least one heteroatom chosen from O, N, P, Si, and S atoms.

4. The block polymer according to claim 3, wherein R is chosen from linear, branched and/or cyclic, saturated and unsaturated hydrocarbon-based radicals optionally comprising a hydrocarbon-based ring that is itself saturated or unsaturated, comprising from 3 to 12 carbon atoms, optionally substituted with at least one entity chosen from ═O, OH, and NH₂ groups and halogen atoms, and/or optionally interrupted with at least one heteroatom chosen from O, N, P, Si and S atoms.

5. The block polymer according to claim 3, in wherein X is chosen from —NH— and —NR— radicals, wherein R is a cyclohexyl.

6. The block polymer according to claim 1, wherein the divalent radical G is chosen from linear, branched and/or cyclic, saturated and unsaturated divalent hydrocarbon-based radicals, optionally comprising a hydrocarbon-based ring that is itself saturated or unsaturated, comprising in total from 2 to 18 carbon atoms, optionally substituted with at least one entity chosen from ═O, OH, and NH₂ groups and halogen atoms, and/or optionally interrupted with at least one heteroatom chosen from O, N, P and Si atoms.

7. The block polymer according to claim 6, wherein G is chosen from linear and branched, saturated divalent hydrocarbon-based radicals optionally comprising a saturated hydrocarbon-based ring, comprising in total from 2 to 18 carbon atoms.

8. The block polymer according to claim 7, wherein G is chosen from linear and branched, saturated divalent hydrocarbon-based radicals optionally comprising a saturated hydrocarbon-based ring, comprising in total from 3 to 10 carbon atoms.

9. The block polymer according to claim 8, wherein G is chosen from ethylene, n-propylene, isopropylene, 1-methylethylene, 2-methylethylene, n-butylene, isobutylene, pentylene, hexylene, cyclohexylene, heptylene, octylene, cyclooctylene, decylene, cyclodecylene, cyclohexyldimethylene, dodecylene and cyclododecylene radicals.

10. The block polymer according to claim 1, wherein P is chosen from the formulae: 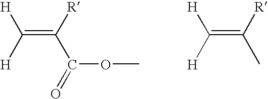
in which R′ is chosen from a hydrogen atom and methyl groups.

11. The block polymer according to claim 1, wherein B is chosen from the divalent aromatic groups of formula (IVa), in which R₁ is chosen from linear, branched and/or cyclic, saturated carbon-based radicals comprising from 1 to 32 carbon atoms.

12. The block polymer according to claim 11, wherein R₁ is chosen from linear, branched and/or cyclic, saturated carbon-based radicals comprising from 3 to 6 carbon atoms

13. The block polymer according to claim 1, wherein the at least one monomer of formula (I) is chosen from one of the following formulae, in which R is chosen from a hydrogen atom and methyl groups:

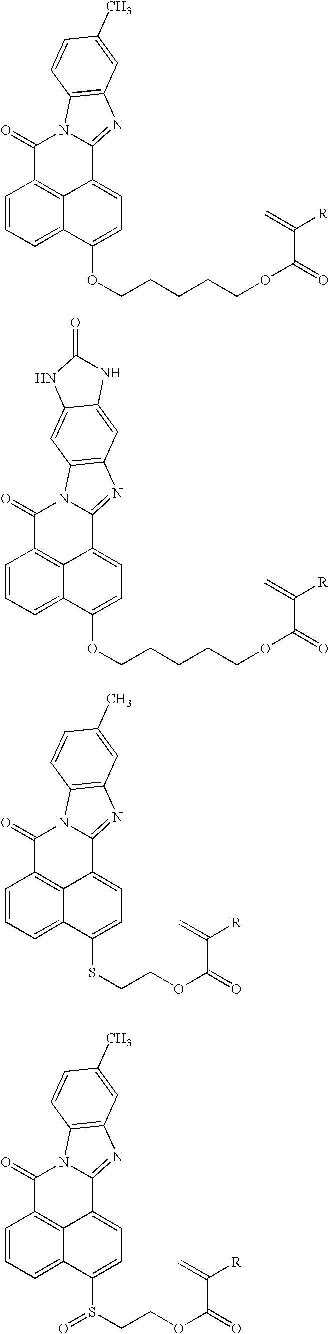

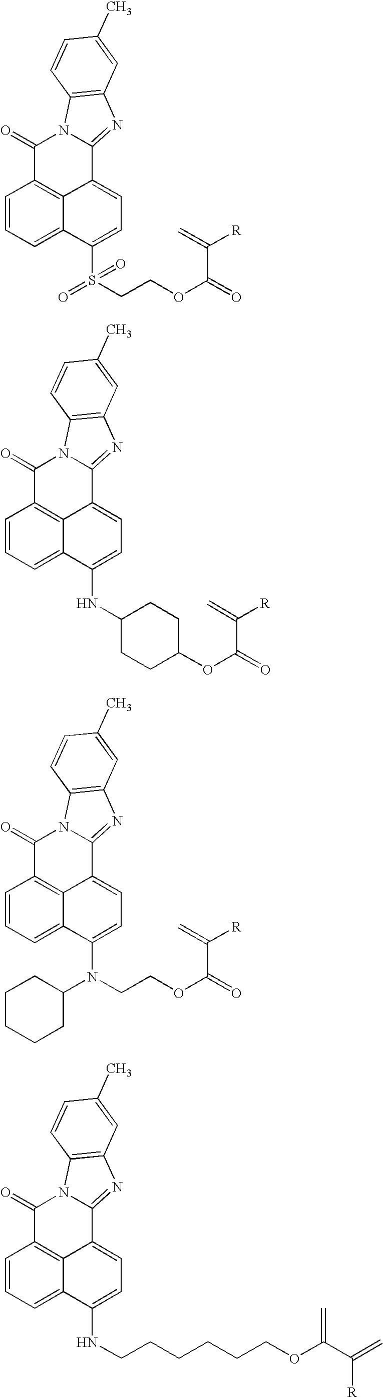

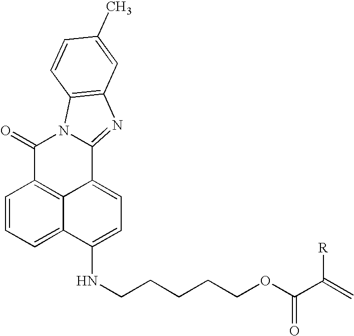

14. The block polymer according to claim 1, further comprising at least one additional monomer chosen from the monomers (i) through (viii):
(i) ethylenic hydrocarbons comprising from 2 to 10 carbons;
(ii) the (meth)acrylates of formulae: 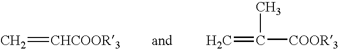
wherein R′₃ is chosen from:
linear and branched alkyl groups of 1 to 18 carbon atoms optionally intercalated with at least one heteroatom chosen from O, N, S and P atoms, the alkyl group also being optionally substituted with at least one substituent chosen from hydroxyl groups, halogen atoms, and groups Si(R₄R₅), in which R₄ and R₅, which may be identical or different, are chosen from C₁ to C₆ alkyl groups and phenyl groups;
C₃ to C₁₂ cycloalkyl groups,
C₃ to C₂₀ aryl groups,
C₄ to C₃₀ aralkyl groups (C₁ to C₈ alkyl),
4- to 12-membered heterocyclic groups comprising at least one heteroatom chosen from O, N and S atoms, the ring being aromatic or non-aromatic,
heterocycloalkyl groups (C1 to C4 alkyl),
wherein the cycloalkyl, aryl, aralkyl, heterocyclic and heterocycloalkyl groups are optionally substituted with at least one substituent chosen from hydroxyl groups, halogen atoms, and linear and branched C₁₋₄ alkyl groups optionally intercalated with at least one heteroatom chosen from O, N, S and P atoms, the alkyl groups also being optionally substituted with at least one substituent chosen from hydroxyl groups, halogen atoms, and groups Si(R₄R₅), in which R₄ and R₅, which may be identical or different, are chosen from C₁ to C₆ alkyl groups and phenyl groups,
R′₃ can also be chosen from —(C₂H₄O)ₘ—R″ groups, with m ranging from 5 to 150, and R″ is chosen from a hydrogen atom and C₁ to C₃₀ alkyl radicals;
(iii) the (meth)acrylamides of formula: 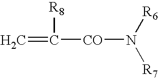
wherein R₈ is chosen from a hydrogen atom and methyl groups; and R₇ and R₆, which may be identical or different, are chosen from:
hydrogen atoms;
linear and branched alkyl groups of 1 to 18 carbon atoms, optionally intercalated with at least one heteroatom chosen from O, N, S and P atoms, the alkyl group also being optionally substituted with at least one substituent chosen from hydroxyl groups, halogen atoms, and groups Si(R₄R₅), in which R₄ and R₅, which may be identical or different, are chosen from C₁ to C₆ alkyl groups and phenyl groups;
C₃ to C₁₂ cycloalkyl groups,
C₃ to C₂₀ aryl groups,
C₄ to C₃₀ aralkyl groups (C₁ to C₈ alkyl group),
4- to 12-membered heterocyclic groups comprising at least one heteroatom chosen from O, N and S atoms, the ring being aromatic or non-aromatic,
heterocycloalkyl groups (C1 to C4 alkyl),
wherein the cycloalkyl, aryl, aralkyl, heterocyclic or heterocycloalkyl groups may be optionally substituted with at least one substituent chosen from hydroxyl groups, halogen atoms, and linear and branched C₁-C₄ alkyl groups optionally intercalated with at least one heteroatom chosen from O, N, S and P atoms, the alkyl groups also being optionally substituted with at least one substituent chosen from hydroxyl groups, halogen atoms, and groups Si(R₄R₅), in which R₄ and R₅, which may be identical or different, are chosen from C₁ to C₆ alkyl groups and phenyl groups;
(iv) the vinyl compounds of formulae:
CH₂═CH—R₉, CH₂═CH—CH₂—R₉ and CH₂═C(CH₃)—CH₂—R₉
wherein R₉ is chosen from hydroxyl groups, halogen atoms, NH₂ groups, OR₁₀ groups in which R₁₀ is chosen from phenyl groups and C₁ to C₁₂ alkyl groups, wherein the monomer is a vinyl or allylic ether; acetamide (NHCOCH₃) groups; groups OCOR₁₁ in which R₁₁ is chosen from linear and branched alkyl groups of 2 to 12 carbons, wherein the monomer is a vinyl or allylic ester; and groups chosen from:
linear and branched alkyl groups of 1 to 18 carbon atoms optionally intercalated with at least one heteroatom chosen from O, N, S and P atoms, the alkyl group also being optionally substituted with at least one substituent chosen from hydroxyl groups, halogen atoms, and groups Si(R4R₅), in which R₄ and R₅, which may be identical or different, are chosen from C₁ to C₆ alkyl groups and phenyl groups;
C₃ to C₁₂ cycloalkyl groups,
C₃ to C₂₀ aryl groups,
C₄ to C₃₀ aralkyl groups (C₁ to C₈ alkyl group),
4- to 12-membered heterocyclic groups comprising at least one heteroatom chosen from O, N and S atoms, the ring being aromatic or non-aromatic, and
heterocycloalkyl groups (C1 to C4 alkyl),
wherein the cycloalkyl, aryl, aralkyl, heterocyclic or heterocycloalkyl groups possibly being optionally substituted with at least one substituent chosen from hydroxyl groups, halogen atoms and linear and branched C1 to C4 alkyl groups optionally intercalated with at least one heteroatom chosen from O, N, S and P atoms, the alkyl groups also being optionally substituted with at least one substituent chosen from hydroxyl groups, halogen atoms, and groups Si(R₄R₅), in which R₄ and R₅, which may be identical or different, are chosen from C₁ to C₆ alkyl groups, and phenyl groups;
(v) (meth)acrylic, (meth)acrylamide and vinyl monomers comprising a fluoro or perfluoro group;
(vi) silicone-based (meth)acrylic, (meth)acrylamide and vinyl monomers;
(vii) ethylenically unsaturated monomers comprising at least one functional group chosen from carboxylic, phosphoric and sulfonic acid, and anhydride functional groups, and the salts thereof; and
(viii) ethylenically unsaturated monomers comprising at least one tertiary amine functional group and salts thereof.

15. The block polymer according to claim 1, further comprising at least one additional monomer chosen from the monomers with an optical effect of formula (A), (B) and/or (C): 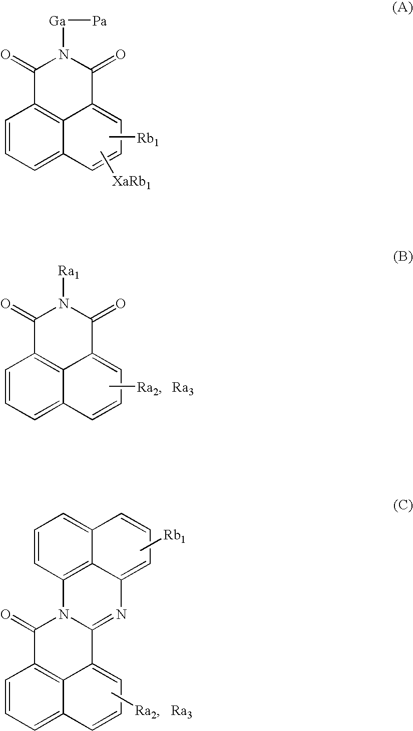
wherein:
Ra₁ is chosen from linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
Rb₁ is chosen from a hydrogen atom, halogen atoms, linear, branched and/or cyclic, saturated and/or unsaturated carbon-based radicals comprising from 1 to 12 carbon-atoms, optionally substituted with at least one group chosen from ═O, OH and NH₂ groups, and/or optionally interrupted with at least one heteroatom chosen from O, N, P, Si and S atoms; and NRR′ groups, wherein R and R′, which may be identical or different, are chosen from a hydrogen atom, and linear, cyclic and/or branched, saturated C₁₋₆ hydrocarbon-based radicals;
Ra₂ and Ra₃, which are present on the same ring or each on a different ring, are independently chosen from hydrogen atoms, halogen atoms, and groups of formula (II): —Xa-Ga-Pa, with the proviso that at least one of the radicals Ra₂ and/or Ra₃ is a group of formula (11), wherein:
Xa is chosen from oxygen and sulfur atoms, and —SO—, —SO₂—, —NH— and —NR₄— radicals, wherein R₄ is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
Ga is chosen from linear, branched and/or cyclic, saturated and/or unsaturated divalent carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
Pa is a polymerizable group chosen from those of formulae (IIIa) to (IIIc): 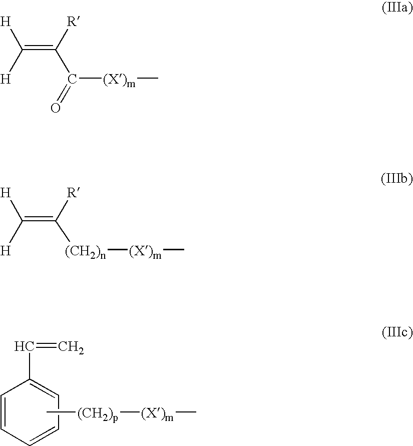
in which:
R′ is chosen from a hydrogen atom and linear or branched, saturated C₁₋₆ hydrocarbon-based radicals,
X′ is chosen from an oxygen atom, and NH and NR″ radicals, wherein R″ is chosen from C₁₋₆ alkyl, C₆₋₁₀aryl, (C₆₋₁₀)aryl(C₁₋₆)alkyl and (C₁₋₆)alkyl(C₆₋₁₀)aryl radicals, the alkyl and/or aryl radicals also being optionally substituted with at least one entity chosen from halogen atoms, and OH, C₁₋₆ alkoxy and C₆₋₁₀ aryloxy groups; and
m is equal to 0 or 1; n is equal to 0 or 1; p is equal to 0, 1 or 2.

16. The block polymer according to claim 1, wherein the at least one monomer of formula (I) is present in an amount ranging from 0.01% to 100% by weight, relative to the weight of the block comprising said at least one monomer.

17. The block polymer according to claim 16, wherein the at least one monomer of formula (I) is present in an amount ranging from 1.5% to 30% by weight, relative to the weight of the block comprising said at least one monomer.

18. The block polymer according to claim 1, wherein the at least one monomer of formula (I) is present in an amount ranging from 0.01% to 70% by weight, relative to the total weight of the polymer.

19. The block polymer according to claim 18, wherein the at least one monomer of formula (I) is present in an amount ranging from 1% to 20% by weight, relative to the total weight of the polymer.

20. A composition comprising, in a physiologically acceptable medium, at least one block polymer comprising at least one first block and at least one second block that are mutually incompatible,
wherein the at least one first and at least one second blocks are linked together via an intermediate segment comprising at least one constituent monomer of the at least one first block and at least one constituent monomer of the at least one second block, and
wherein at least one of the blocks comprises at least one monomer of formula (I): 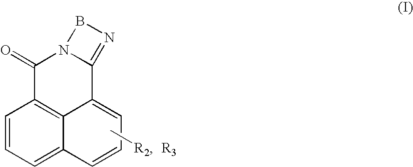
wherein:
R₂ and R₃, which are present on the same ring or each on a different ring, are independently chosen from hydrogen atoms, halogen atoms, and groups of formula (II): —X-G-P, with the proviso that at least one of the radicals R₂ and/or R₃ is a group of formula (II), in which:
X is chosen from oxygen and sulfur atoms, and —SO—, —SO₂—, —NH— and —NR— radicals wherein R is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
P is a polymerizable group chosen from those of formulae (IIIa) to (IIIc): 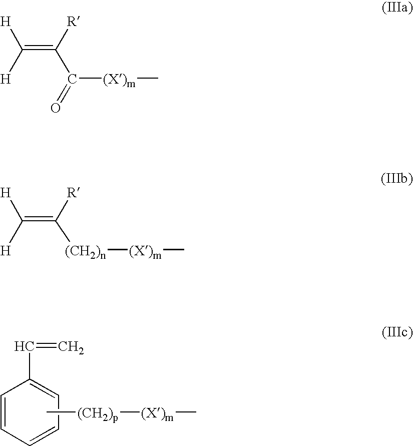
wherein:
R′ is chosen from a hydrogen atom and linear and branched, saturated C₁₋₆ hydrocarbon-based radicals,
X′ is chosen from oxygen atoms, and NH and NR″ groups, wherein R″ is chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, the alkyl and/or aryl radicals also possibly being substituted with at least one entity chosen from halogen atoms, and OH, C₁₋₆ alkoxy and C₆₋₁₀ aryloxy groups;
m is equal to 0 or 1; n is equal to 0 or 1; p is equal to 0, 1 or 2;
B is a divalent aromatic group chosen from those of formulae (IVa) to (IVd): 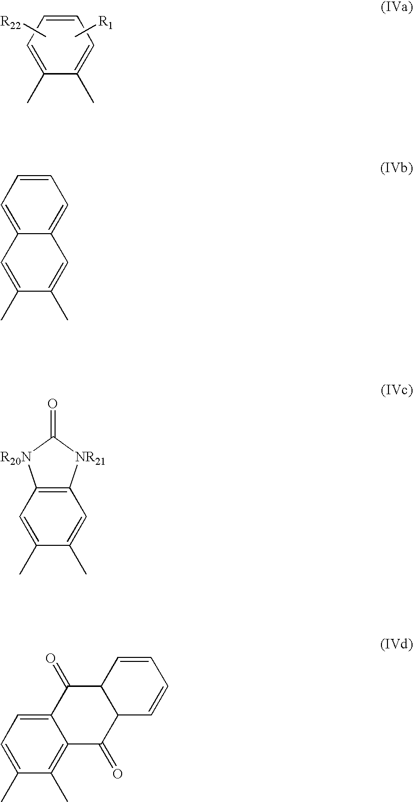
wherein:
R₁ is chosen from linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₂ is chosen from a hydrogen atom, and linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₀ and R₂₁, which may be identical or different, are chosen from hydrogen atoms, linear and branched C₁₋₈ alkyl radicals, and cyclopentyl, cyclohexyl, cyclooctyl, cyclodecyl, cyclododecyl, benzyl, naphthyl and phenyl radicals.

21. The composition according to claim 20, wherein the at least one block polymer is present in an amount ranging from 0.01% to 75% by weight, relative to the total weight of the composition.

22. The composition according to claim 21, wherein the at least one block polymer is present in an amount ranging from 6% to 25% by weight, relative to the total weight of the composition.

23. The composition according to claim 20, wherein the physiologically acceptable medium comprises a hydrophilic medium comprising water, or a mixture of water and at least one hydrophilic organic solvent, and/or comprises a fatty phase.

24. The composition according to claim 23 wherein the fatty phase comprises at least one component chosen from waxes, pasty fatty substances, gums, lipophilic organic solvents and oils.

25. The composition according to claim 20, further comprising a particulate phase, comprising at least one of pigments and/or nacres and/or fillers.

26. The composition according to claim 20, further comprising at least one dyestuff chosen from water-soluble dyes and/or liposoluble dyes.

27. The composition according to claim 20, comprising at least one additional polymer.

28. The composition according to claim 27, wherein the at least one additional polymer is a film forming polymer.

29. The composition according to claim 20, further comprising at least one ingredient chosen from vitamins, thickeners, gelling agents, trace elements, softeners, sequestering agents, fragrances, acidifying and basifying agents, preserving agents, sunscreens, surfactants, antioxidants, agents for preventing hair loss, antidandruff agents, propellants and ceramides.

30. The composition according to claim 20, wherein the composition is in the form of a suspension; a dispersion; an optionally thickened or gelled oily solution; an oil-in-water, water-in-oil or multiple emulsion; a gel; a mousse; an oily or emulsified gel; a dispersion of vesicles; a two-phase or multiphase lotion; a spray; a free, compact or cast powder; an anhydrous paste; a lotion; a cream; a salve; a soft paste; an ointment; a cast solid; a molded solid; and a compacted solid.

31. The composition according to claim 20, wherein the composition is in the form of a care and/or makeup product for bodily or facial skin, the lips, the nails, the eyelashes, the eyebrows and/or the hair, an antisun product, a self-tanning product; or a hair product for caring for, treating, shaping, making up or coloring the hair.

32. The composition according to claim 31, which is in the form of a makeup composition chosen from a foundation, a makeup rouge, an eyeshadow; a lipstick, a lipcare product; a concealer product; a blusher, a mascara, an eyeliner; an eyebrow makeup product; a lip pencil; an eye pencil; a nail varnish; a nailcare product; a body makeup product; a hair makeup product; a protective or care composition for the skin of the face, the neck, the hands or the body; a moisturizing or treating composition; an antisun composition; an artificial tanning composition; and a hair product chosen from products for coloring, holding the hairstyle, shaping the hair, caring for, treating and cleansing the hair.

33. A cosmetic treatment process for making up or caring for keratin materials, comprising
applying to the keratin materials a cosmetic composition comprising, in a physiologically acceptable medium, at least one block polymer comprising at least one first block and at least one second block that are mutually incompatible,
wherein the at least one first and at least one second blocks are linked together via an intermediate segment comprising at least one constituent monomer of the at least one first block and at least one constituent monomer of the at least one second block, and
wherein at least one of the blocks comprises at least one monomer of formula (I): 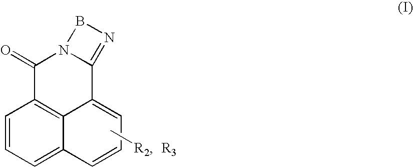
wherein:
R₂ and R₃, which are present on the same ring or each on a different ring, are independently chosen from hydrogen atoms, halogen atoms, and groups of formula (II): —X-G-P, with the proviso that at least one of the radicals R₂ and/or R₃ is a group of formula (II), in which:
X is chosen from oxygen and sulfur atoms, and —SO—, —SO₂—, —NH— and —NR— radicals wherein R is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms optionally substituted with at least one entity chosen from ═O, OH, and NH₂ groups and halogen atoms, and/or optionally interrupted with at least one heteroatom chosen from O, N, P, Si and S atoms;
P is a polymerizable group chosen from those of formulae (IIIa) to (IIIc): 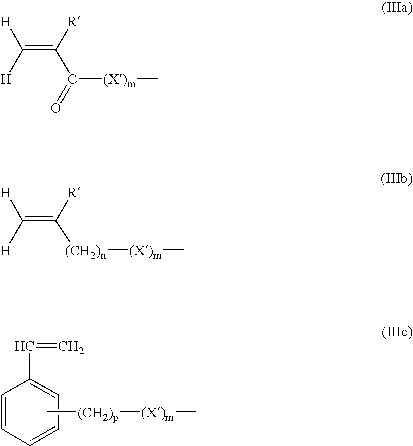
wherein:
R′ is chosen from a hydrogen atom and linear and branched, saturated C₁₋₆ hydrocarbon-based radicals,
X′ is chosen from an oxygen atom, and NH and NR″ groups, wherein R″ is chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, the alkyl and/or aryl radicals also possibly being substituted with at least one entity chosen from halogen atoms, and OH, C₁₋₆ alkoxy and C₆₋₁₀ aryloxy groups;
m is equal to 0 or 1; n is equal to 0 or 1; p is equal to 0, 1 or 2;
B is a divalent aromatic group chosen from those of formulae (IVa) to (IVd): 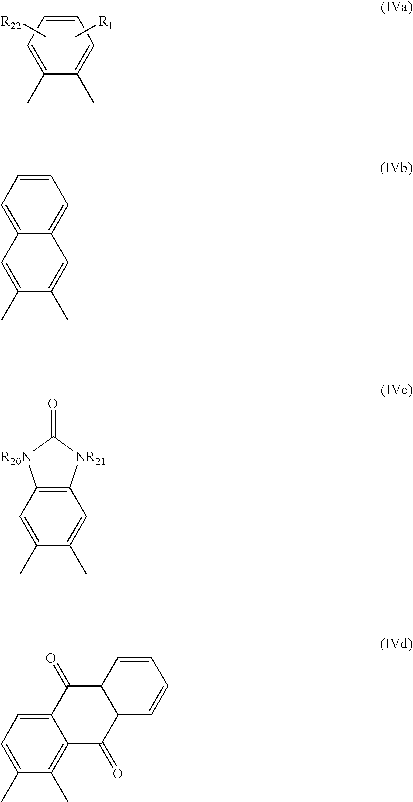
wherein:
R₁ is chosen from linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₂ is chosen from a hydrogen atom, and linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₀ and R₂₁, which may be identical or different, are chosen from hydrogen atoms, linear and branched C₁₋₈ alkyl radicals, and cyclopentyl, cyclohexyl, cyclooctyl, cyclodecyl, cyclododecyl, benzyl, naphthyl and phenyl radicals.

34. A nail varnish composition comprising at least one organic solvent, at least one film-forming polymer, at least one block polymer comprising at least one first block and at least one second block that are mutually incompatible,
wherein the at least one first and at least one second blocks are linked together via an intermediate segment comprising at least one constituent monomer of the at least one first block and at least one constituent monomer of the at least one second block, and
wherein at least one of the blocks comprises at least one monomer of formula (I): 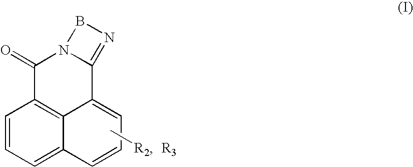
wherein:
R₂ and R₃, which are present on the same ring or each on a different ring, are independently chosen from hydrogen atoms, halogen atoms, and groups of formula (II): —X-G-P, with the proviso that at least one of the radicals R₂ and/or R₃ is a group of formula (II), in which:
X is chosen from oxygen and sulfur atoms, and —SO—, —SO₂—, —NH— and —NR— radicals wherein R is chosen form linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
P is a polymerizable group chosen from those of formulae (IIIa) to (IIIc): 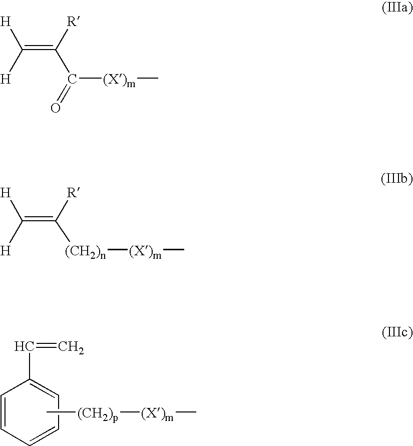
wherein:
R′ is chosen from a hydrogen atom and linear and branched, saturated C₁₋₆ hydrocarbon-based radicals,
X′ is chosen from oxygen atoms, and NH and NR″ groups, wherein R″ is chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, the alkyl and/or aryl radicals also possibly being substituted with at least one entity chosen from halogen atoms, and OH, C₁₋₆ alkoxy and C₆₋₁₀ aryloxy groups;
m is equal to 0 or 1; n is equal to 0 or 1; p is equal to 0, 1 or 2;
B is a divalent aromatic group chosen from those of formulae (IVa) to (IVd): 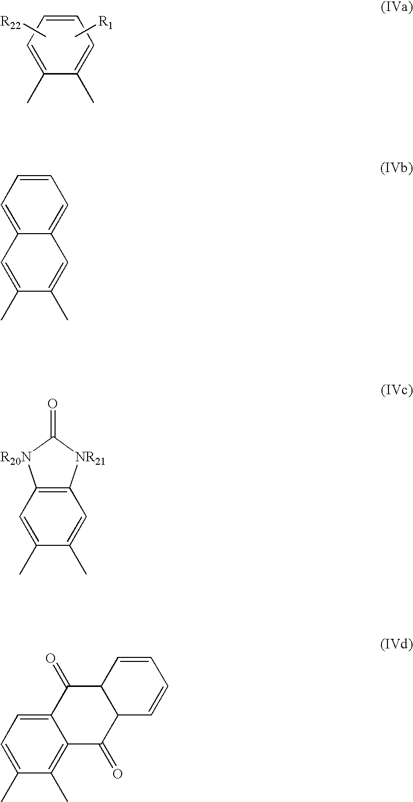
wherein:
R₁ is chosen from linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₂ is chosen from a hydrogen atom, and linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₀ and R₂₁, which may be identical or different, are chosen from hydrogen atoms, linear and branched C₁₋₈ alkyl radicals, and cyclopentyl, cyclohexyl, cyclooctyl, cyclodecyl, cyclododecyl, benzyl, naphthyl and phenyl radicals;
and optionally at least one pigment and/or at least one dye.

35. A foundation composition comprising at least one oil in a fatty phase, at least one pigment, optionally an aqueous phase, and at least one block polymer comprising at least one first block and at least one second block that are mutually incompatible,
wherein the at least one first and at least one second blocks are linked together via an intermediate segment comprising at least one constituent monomer of the at least one first block and at least one constituent monomer of the at least one second block, and
wherein at least one of the blocks comprises at least one monomer of formula (I): 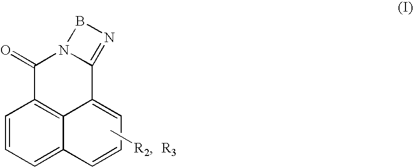
wherein:
R₂ and R₃, which are present on the same ring or each on a different ring, are independently chosen from hydrogen atoms, halogen atoms, and groups of formula (II): —X-G-P, with the proviso that at least one of the radicals R₂ and/or R₃ is a group of formula (II), in which:
X is chosen from oxygen and sulfur atoms, and —SO—, —SO₂—, —NH— and —NR— radicals wherein R is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
P is a polymerizable group chosen from those of formulae (IIIa) to (IIIc): 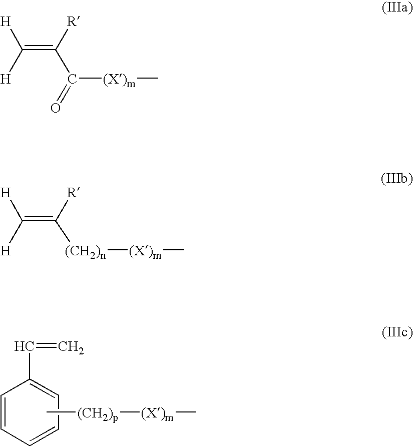
wherein:
R′ is chosen from a hydrogen atom and linear and branched, saturated C₁₋₆ hydrocarbon-based radicals,
X′ is chosen from an oxygen atom, and NH and NR″ groups, wherein R″ is chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, the alkyl and/or aryl radicals also possibly being substituted with at least one entity chosen from halogen atoms, and OH, C₁₋₆ alkoxy and C₆₋₁₀ aryloxy groups;
m is equal to 0 or 1; n is equal to 0 or 1; p is equal to 0, 1 or 2;
B is a divalent aromatic group chosen from those of formulae (IVa) to (IVd): 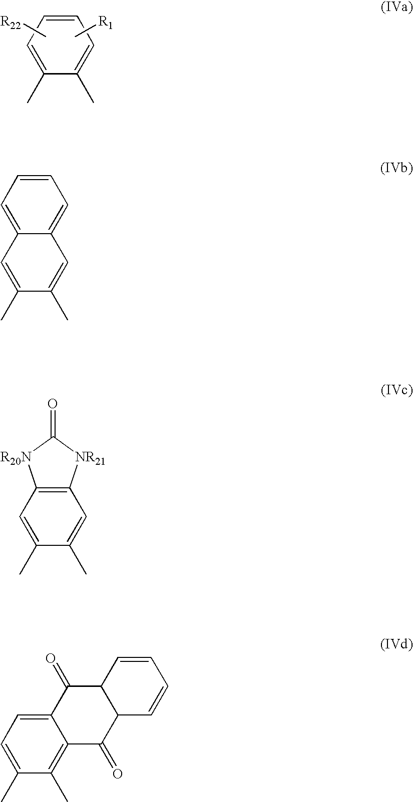
wherein:
R₁ is chosen from linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₂ is chosen from a hydrogen atom, and linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₀ and R₂₁, which may be identical or different, are chosen from hydrogen atoms, linear and branched C₁₋₈ alkyl radicals, and cyclopentyl, cyclohexyl, cyclooctyl, cyclodecyl, cyclododecyl, benzyl, naphthyl and phenyl radicals.

36. An anti-ageing or anti-wrinkle composition comprising, in an amount sufficient to reduce the-effects of age or wrinkles on skin, at least one block polymer comprising, at least one first block and at least one second block that are mutually incompatible,
wherein the at least one first and at least one second blocks are linked together via an intermediate segment comprising at least one constituent monomer of the at least one first block and at least one constituent monomer of the at least one second block, and
wherein at least one of the blocks comprises at least one monomer of formula (I): 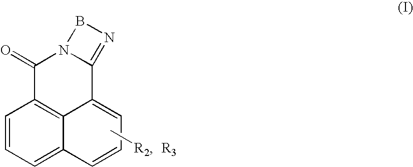
wherein:
R₂ and R₃, which are present on the same ring or each on a different ring, are independently chosen from hydrogen atoms, halogen atoms, and groups of formula (II): —X-G-P, with the proviso that at least one of the radicals R₂ and/or R₃ is a group of formula (II), in which:
X is chosen from oxygen and sulfur atoms, and —SO—, —SO₂—, —NH— and —NR— radicals wherein R is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
P is a polymerizable group chosen from those of formulae (IIIa) to (IIIc): 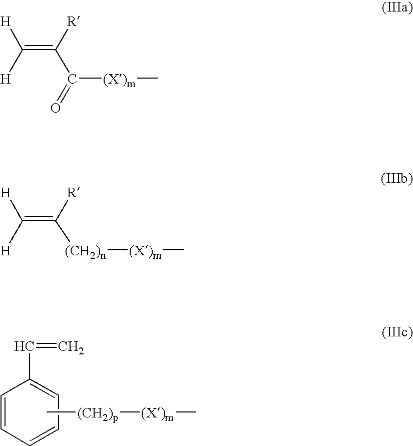
wherein:
R′ is chosen from a hydrogen atom and linear and branched, saturated C₁₋₆ hydrocarbon-based radicals,
X′ is chosen from an oxygen atom, and NH and NR″ groups, wherein R″ is chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, the alkyl and/or aryl radicals also possibly being substituted with at least one entity chosen from halogen atoms, and OH, C₁₋₆ alkoxy and C₆₋₁₀ aryloxy groups;
m is equal to 0 or 1; n is equal to 0 or 1; p is equal to 0, 1 or 2;
B is a divalent aromatic group chosen from those of formulae (IVa) to (IVd): 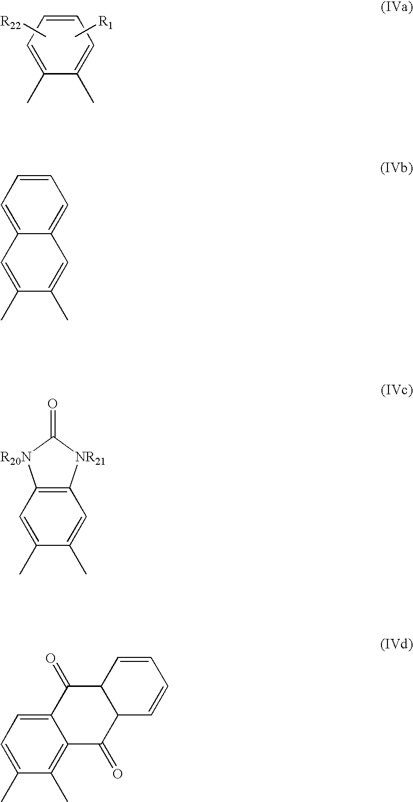
wherein:
R₁ is chosen from linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₂ is chosen from a hydrogen atom, and linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₀ and R₂₁, which may be identical or different, are chosen from hydrogen atoms, linear and branched C₁₋₈ alkyl radicals, and cyclopentyl, cyclohexyl, cyclooctyl, cyclodecyl, cyclododecyl, benzyl, naphthyl and phenyl radicals.

37. A cosmetic process for treating wrinkled skin, comprising applying to the skin a cosmetic composition comprising, in a cosmetically acceptable medium, at least one block polymer comprising at least one first block and at least one second block that are mutually incompatible,
wherein the at least one first and at least one second blocks are-linked together via an intermediate segment comprising at least one constituent monomer of the at least one first block and at least one constituent monomer of the at least one second block, and
wherein at least one of the blocks comprises at least one monomer of formula (I): 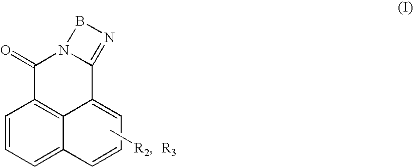
wherein:
R₂ and R₃, which are present on the same ring or each on a different ring, are independently chosen from hydrogen atoms, halogen atoms, and groups of formula (II): —X-G-P, with the proviso that at least one of the radicals R₂ and/or R₃ is a group of formula (II), in which:
X is chosen from oxygen and sulfur atoms, and —SO—, —SO₂—, —NH— and —NR— radicals wherein R is chosen from linear, branched and/or cyclic, saturated and/or unsaturated carbon-based radicals comprising from 1 to 30 carbon atoms, optionally substituted with at least one entity chosen from ═O, OH, and NH₂ groups and halogen atoms, and/or optionally interrupted with at least one heteroatom chosen from O, N, P, Si and S atoms;
G is chosen from linear, branched and/or cyclic, saturated and/or unsaturated divalent carbon-based radicals comprising from 1 to 32 carbon atoms optionally substituted with at least one entity chosen from ═O, OH, and NH₂ groups and halogen atoms, and/or optionally interrupted with at least one heteroatom chosen from O, N, P, Si and S atoms;
P is a polymerizable group chosen from those of formulae (IIIa) to (IIIc): 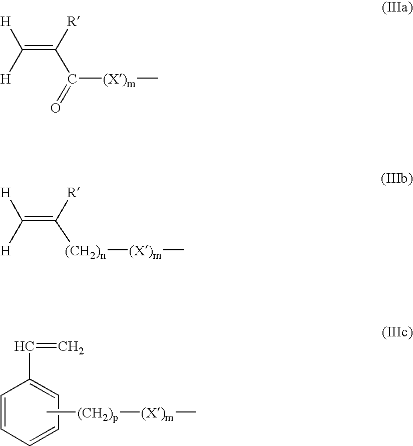
wherein:
R′ is chosen from a hydrogen atom and linear and branched, saturated C₁₋₆ hydrocarbon-based radicals,
X′ is chosen from an oxygen atom, and NH and NR″ groups, wherein R″ is chosen from C₁₋₆ alkyl, C₆₋₁₀ aryl, (C₆₋₁₀)aryl(C₁₋₆)alkyl and (C₁₋₆)alkyl(C₆₋₁₀)aryl radicals, the alkyl and/or aryl radicals also possibly being substituted with at least one entity chosen from halogen atoms, and OH, C₁₋₆ alkoxy and C₆₋₁₀ aryloxy groups;
m is equal to 0 or 1; n is equal to 0 or 1; p is equal to 0, 1 or 2;
B is a divalent aromatic group chosen from those of formulae (IVa) to (IVd): 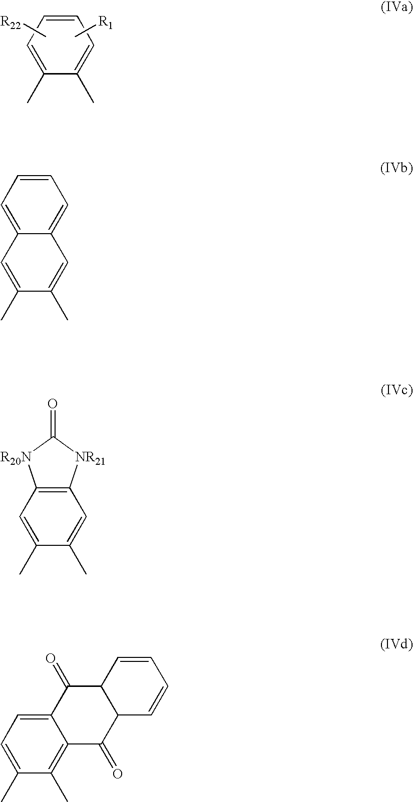
wherein:
R₁ is chosen from linear, branched and/or cyclic, saturated and/or unsaturated carbon-based radicals comprising from 1 to 32 carbon atoms optionally substituted with at least one entity chosen from ═O, OH, and NH₂ groups and halogen atoms, and/or optionally interrupted with at least one heteroatom chosen from O, N, P, Si and S atoms;
R₂₂ is chosen from a hydrogen atom, and linear, branched and/or cyclic, saturated and/or unsaturated carbon-based radicals comprising from 1 to 32 carbon atoms, optionally substituted with at least one entity chosen from ═O, OH, and NH₂ groups and halogen atoms, and/or optionally interrupted with at least one heteroatom chosen from O, N, P, Si and S atoms;
R₂₀ and R₂₁, which may be identical or different, are chosen from hydrogen atoms, linear and branched C₁₋₈ alkyl radicals, and cyclopentyl, cyclohexyl, cyclooctyl, cyclodecyl, cyclododecyl, benzyl, naphthyl and phenyl radicals.
